(19) United States
(12) Patent Application Publication
(10) Pub. No.: US 2015/0284689 A1
(43) Pub. Date: Oct. 8, 2015

(54) STRATEGY FOR ENGINEERING VARIOUS 3D TISSUES, ORGANOIDS AND VASCULATURE

(71) Applicant: THE REGENTS OF THE UNIVERSITY OF CALIFORNIA, Oakland, CA (US)

(72) Inventor: Sanjay K. Nigam, Del Mar, CA (US)

(21) Appl. No.: 14/438,334

(22) PCT Filed: Oct. 24, 2013
(86) PCT No.: PCT/US13/66632

§ 371 Date: Apr. 24, 2015

Related U.S. Application Data
(60) Provisional application No. 61/719,151, filed on Oct. 26, 2012.

(51) Int. Cl. C12N 5/071; A61L 27/36

(52) U.S. Cl. CPC C12N 5/0697; A61L 27/3604; A61L 27/3625; C12N 2501/113; C12N 2501/117; C12N 2501/13; C12N 2501/10; C12N 2501/12; C12N 2501/148; C12N 2501/16; C12N 2501/15; C12N 2501/415; C12N 2501/42; C12N 2501/00; C12N 2502/09; C12N 2502/13; C12N 2533/54; C12N 2533/30; C12N 2533/78; C12N 2533/70

(57) ABSTRACT

Provided are methods and compositions for constructing stable mammalian epithelial and/or endothelial tissues and organs. Disclosed are methods of using active epithelial and/or endothelial growth factors having the capability of effectuating induction of growth and morphogenesis of cells.

### CROSS REFERENCE TO RELATED APPLICATIONS

This application claims priority under 35 U.S.C. §119 to U.S. Provisional Application Ser. No. 61/719,151, filed Oct. 26, 2012, the disclosure of which is incorporated herein by reference.

### FIELD OF THE INVENTION

The invention generally concerns methods of tissue engineering, and more particularly relates to methods and compositions for generating various 3D tissues, organoids and vasculature.

### BACKGROUND

There are a number of branched tubular organs in the body including, but not limited to, the lungs, pancreas, kidneys and various glands. Tissue replacement and tissue reconstruction could benefit greatly by providing engineered sources of these branched tubular organs.

### SUMMARY

The disclosure describes a “generic” branching ductal “cellular scaffold” (with its own secreted matrix) that can be used as a fundamental building block structure in which any number of epithelial and/or endothelial branching organs can be built around. The particular organ produced is largely determined by which “poised” organ-specific mesenchymal cells are made available. Examples of organ, organoid, or tissue which can be produced by the methods of the disclosure include, but are not limited to, thyroid, pancreas, ureters, bladder, urethra, adrenal glands, lung, liver, pineal gland, pituitary gland, parathyroid glands, thymus gland, adrenal glands, appendix, gallbladder, spleen, prostate gland, reproductive organs, and vascular tissue.

From one branching structure, hundreds, thousands or maybe millions of different branching epithelial and/or endothelial organs can be created. The epithelial and/or endothelial organ is built around or out of these propagate-able three dimensional (3D) iterative tip-stalk generators while the mesenchyme determines, through cell contact: and short-acting factors, organ-specific features. Thus, the 3D branching ductal structure can be viewed, as a “cellular scaffold” that can generate the relatively insoluble dynamic matrix scaffold. The in vitro developmental biology is quite clear and directly correlative with the in vivo biology. In other words, the disclosure provides that from a relatively small number of cells a 3D tip-stalk generator can be differentiated, which then though a morphogenetic process (e.g., lumen formation and expansion, tubulogenesis, branching morphogenesis, mesenchymal-epithelial transitions (MET) and epithelial-mesenchymal transitions (EMT)) a large number of independent branching ductal “cellular scaffolds” (with matrix) can be created, and from which, when interfaced with mesenchyme, an independent organ can be generated from each “cellular scaffold.” In a particular embodiment, a large number of independent branching ductal “cellular scaffolds” (with matrix) can be created by using a branching morphogenesis process. In an alternate embodiment, a large number of independent branching ductal “cellular scaffolds” (with matrix) can be created by a tubulogenesis process. In a further embodiment, a large number of independent branching ductal “cellular scaffolds” (with matrix) can be created by a MET or EMT process.

As described in the literature, kidney branching ducts (ureteric bud) secreted surfactant proteins when recombined with embryonic lung mesenchyme. The mesenchyme, therefore, provides a key and determinative role in defining the organ-specific features of the developing organ. Thus, one can imagine, following the protocols outlined herein, a near inexhaustible and replenishable source of “cellular scaffold” material can produce branching ductal systems, which can then be recombined at-will with the appropriate type of mesenchyme (e.g., lung, kidney, salivary gland), including from patient-derived cells, to create each organ. In a particular embodiment, the mesenchyme can be embryonic mesenchyme. In an alternate embodiment, the mesenchyme is an organ-specific mesenchyme differentiated from iPS cells, embryonic fibroblasts or cells derived from bone marrow or cord blood. In a further embodiment, the mesenchyme is an organ-specific mesenchyme differentiated from iPS cells. In vet a further embodiment, the mesenchyme is an organ-specific mesenchyme differentiated from embryonic fibroblasts. In another embodiment, the mesenchyme is an organ-specific mesenchyme differentiated from bone marrow or cord blood.

The proto-organs generated by using the protocols described herein can be used in a variety of applications, including as surrogate organs, tissue reservoirs for organ tissue, or as models to test the effects of various agents on organ function. For example, in patients that suffer from organ failure (e.g., Pancreatic insufficiency or chronic kidney failure) the proto-organs of the disclosure can implanted as a surrogate organs, or alternatively the proto-organs could be used in toxicity studies to analyze the effects of drugs and environmental toxins on the organ's function. Ultimately, the infrastructure required to perform the procedures of the disclosure is minimal, and can be performed in a medium-sized clinical, biotech or pharmaceutical lab or in a core facility at a research institution.

In a particular embodiment, the disclosure provides for a method of generating an organ specific tubular tissue structure, comprising: (a) contacting a stem cell, branching epithelial cell, or branching endothelial cell with one or more cell survival agents or biological active agents to stimulate growth and proliferation; (b) contacting the cells with one or more branching agents that promote formation of tubular tissue branches and/or globular morphology to generate budding tissue; (c) combining the bud tissue with tissue specific mesenchyme in a biocompatible matrix; and (d) culturing the combination to form an organ specific tubular tissue structure in vitro. In a further embodiment, the branching epithelial cell is a ureteric bud, bud or duct of a salivary gland, Wolffian duct bud, or ureteric and Wolffian duct bud tissue. In yet another embodiment, the organ specific mesenchyme is from the group consisting of breast, pancreatic, lung, gall bladder, spleen, liver, reproductive, and glandular mesenchyme. In a further embodiment, the glandular mesenchyme is selected from the group consisting of adrenal, salivary, prostate, thymus, parathyroid, pituitary, and pineal mesenchyme. In another embodiment, the organ specific tubular tissue structure comprises a tissue type selected from thyroid, pancreas, ureters, bladder, urethra, adrenal glands, lung, liver, pineal gland, pituitary gland, parathyroid glands, thymus gland, adrenal glands, appendix, gallbladder, spleen, prostate gland, and reproductive organs. In another embodiment, the organ specific tubular tissue structure comprises kidney tissue.

In a certain embodiment, the disclosure provides for one or more cell survival agents selected from FGF1, FGF7 and a combination thereof either alone or in combination with one or more of GDNF, PTN, HRG, or BSN-CM. In another embodiment, the one or more growth agents are selected from FGF1, FGF7, PTN, GDNF, BSN-CM HRG and BSN. In a further embodiment, the one or more cell survival agents are selected from FGF1, FGF7, FGF1 and FGF7, PTN and GDNF, FGF1 and GDNF, FGF7 and GDNF, BSN-CM and FGF1, HRG and FGF1, PTN and FGF1, BSN and FGF7, HRG and FGF7, PTN and FGF7, BSN and FGF1 and GDNF, HRG and FGF1 and GDNF, PTN and FGF1 and GDNF, BSN and FGF7 and GDNF, HRG and FGF7 and GDNF, and PTN and FGF7 and GDNF. In a particular embodiment, the one or more branching and/or growth factor agents are selected from FGF1, FGF2FGF3, FGF4, EGF5, FGF6, FGF7, FGF8, FGF9, FGF10, FGF15, FGF16FGF17, FGF18, FGF19, FGF20, FGF21, FGF22, FGF23, EGF, TGFα, HGF, heparin-binding EGF-like growth factor (HB-EFG), amphirgulin (AR), betacellulin (BTC) , epiregulin (EPR), epigen ARF4, CAV1, CAV3, CBL, CBLB, CBLC, CDC25A, CRK, CTNNB1, DCN, GRB14, grb2, ,JAK2, MUC1, NCK1, NCK2, PKC-α, PLCG1, PLSCR1, PTPN1, PTPN11, PTPN6, PTPRK, SH2D3A, SH3KBP1, SHC1, SOS1, src STAT1, STAT3, STAT5A, UBC, WAS, activin, VEGF, BMP4, TGFβ1, TGFβ2, TGFβ3, gremlini, ErbB/neuregulin/heregulin, EGFR, FGFR2, PAX2, EYA1, SIX1, WNT4, WNT5a, WNT11, PTN, TGEα, GDNF, PDGF, bFGF, IGF-1, β-catenin, PEA3/ETV4, Hs2st, MMP2, MT1-MMP, fibronectin, Notch, Sonic Hedgehog, Sproutyl, Sprouty2, SFRP1, semaphorins, Slit/Robo, and EPH/Ephrin.

In a certain embodiment, the disclosure provides for biocompdtible matrix comprising one or more materials selected from hyaluronic acid, entactin, cotton, collagen, polyglycolic acid, cat gut suture, cellulose, gelatin, dextran, polyamide, polyester, polystyrene, polypropylene, polyacrylate, polyvinyl, polycarbonate, polytetxafluorethylene, nitrocellulose compound, and Matrigel. In a further embodiment, the biocompatible matrix is treated to contain proteoglycans, Type I collagen, Type IV collagen, laminin, proteoglycans, fibronectin, or combinations thereof.

In another embodiment, the disclosure provides for breast; pancreatic; glandular, including adrenal, salivary, prostate, thymus, parathyroid, pituitary, and pineal; lung; gall bladder; spleen; liver; reproductive; or vascular tissue developed by the methods disclosed herein. In an alternate embodiment, the disclosure provides for kidney tissue developed by the methods disclosed herein. In a further embodiment, the tissue is implanted into a subject so as to induce vascularization of the tissue. In a further embodiment, a disease, disorder or condition in a subject can be treated by implanting a tissue described herein or a portion thereof in a subject. In yet a further embodiment, the disease, disorder or condition is selected from the group consisting of Sjogren syndrome, Addison's disease, Celiac disease, chronic thyroiditis, multiple sclerosis, systemic lupus erythematosus, diabetes, pancreatitis, hypertension, chronic kidney disease, polycystic kidney disease, end stage renal disease, malignant hypertension, acute liver failure, chronic liver failure, chronic hepatitis infection, liver cirrhosis, hemochromatosis, Wilson's disease, nonalcoholic steatohepatitis, hepatocellular carcinoma, hepatoblastoma, cholangiocarcinoma, biliary artesia, coronary artery disease, cardiomyopathy, heart failure, cystic fibrosis, emphysema, obstructive lung disease, short bowel syndrome, necrotizing enterocolitis, and Crohn's disease. In another embodiment, the disclosure provides a method to reconstruct tissue that has been removed from a subject comprising: implanting a tissue or a portion of the tissue made by the methods described herein in a subject to replace or reconstruct damaged tissue (e.g., damage resulting from diseases, age-related damage or natural deterioration of tissue due to age, damage resulting from accidents, and damage resulting front the environment). In a particular embodiment, breast tissue created by the methods disclosed herein can be used to replace or reconstruct breast tissue which has been removed via a mastectomy. In a certain embodiment, the disclosure provides for a test kit comprising a tissue described herein.

In a particular embodiment, the disclosure provides a method of generating a tubular tissue structure, comprising: contacting a stem cell or branching epithelial cell, with a cell survival agent or biological active agent to stimulate growth and proliferation; (b) contacting the cells with a branching agent that promotes formation of tubular tissue branches and/or globular morphology to generate budding tissue; (c) culturing the budding tissue in vitro under conditions that induce branching morphogenesis to generate a population of tubular branches, subdividing the population of tubular branches, and re-suspending each subpopulation in culture media and repeating; (d) combining the tubular branches with tissue specific mesenchyme in a biocompatible matrix; and (e) culturing the combination to form a branched tissue specific organ in vitro.

In a particular embodiment, the disclosure provides a method comprising: differentiating stem cells to form tissue specific mesenchymal cells; differentiating stem cells to form epithelial bud cells; combining the cells in a biocompatible matrix or gel; and culturing the combination to form a specific tissue.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 provides a schematic of the of the budding and organ differentiation process that allows for the generation of various organs that can then be utilized to treat a large variety of diseases, conditions, or disorders, or alternatively can be used to reconstruct damaged organs. Disorders provided in parentheses represent only a small sampling of the diseases, conditions, or disorders treatable by the processes disclosed herein.

FIG. 2 presents a diagram which shows that when primary cells are exposed to certain factors, specific types of tissue develop. As shown, when mouse embryonic fibroblasts come into contact with Hnf4a (Hnf1α), late kidney mesenchyme/pre-proximal tubule tissue forms; and when induced pluripotent stem cells are exposed to OSRI, intermediate mesoderm forms, while when the same induced pluripotent stem cells when exposed to BMP, forms pre-proximal tubule tissue.

FIG. 3 provides a diagram to demonstrate the general applicability of the processes of the disclosure to generate a variety of organs and tissues based upon inducing branching of an isolated bud (e.g., ureteric bud) and the selection of an organic-specific mesenchyme. As shown, a bud of nonspecific origin can be induced to undergo branching and when combined with an organ-specific mesenchyme eventually leads to the production of a specific organ or tissue (e.g., kidney, lung, various glands, and pancreas).

FIG. 4 provides a schematic of various approaches to in vitro engineer various organ-like tissues. First, an epithelial or endothelial bud or duct is isolated from a biopsy (e.g., from a kidney, salivary, or other gland biopsy) and induced to undergo branching. The branched in vitro-formed tubule is then recombined with cluster of mesenchyme cells, which can originate from any number of tissues. After a few days or weeks of mutual induction, the recombined tissue will resemble the target tissue (e.g., kidney-like tissue). The possibility of using cells (iPSCs) to engineer branching tubules and/or varic)us mesenchyme-like tissues is also indicated.

FIG. 5 demonstrates that ureteric bud (GB) can undergo branching morphogenesis in vitro in the presence of soluble factors (without contact with mesenchyme.) As shown, at day 0: UB with only on branch point; at day 3: UB which has formed multiple branch points; and at day 12: UB has formed an extensively branched network.

FIG. 6 provides a schematic of the developmental approaches to in vitro engineer organ tissue.

FIG. 7A-I provides a diagram of the development of the metanephric kidney from its progenitor tissues, the ureteric bud (UB) and the metanephric mesenchyme (MM). (A): Metanephric kidney development is initiated with the outgrowth of the UB from the Wolffian duct/pronephric duct and its penetrance into the MM in response to soluble factors (e.g., glial-derived neurotropic factor) elaborated by this aggregation of intermediate mesoderm derived cells. (B-D): A macroscopic view of kidney collecting system development through PB branching morphogenesis within the MM. (E-I): Depiction of nephron development from the MM at the tips of the branching UB.

FIG. 8A-F provides a schematic showing the generation of various tissues structures leading ultimately to a tissue engineered kidney. (A) From Wolffian duct cells a Wolffian duct is recreated. (B) The recreated Wolffian duct is then is induced to bud. (C) A ureteric bud (UB) is formed from PB cells or isolated from WB. (D) The PE is induced to branch and combined with metanephric mesenchyme (MM) cells in a 3D hydrogel. (B) After a week, a kidney proto-organ has formed. (F) After more than a week, an engineered kidney has resulted.

FIG. 9 provides for the ex vivo propagation of isolated ureteric buds (UBs). (A, B) Phase contrast photomicrographs of isolated UBs cultured in 3D extracellular matrix (ECM) gels in the presence of BSN-condition media supplemented with 10% fetal calf serum, 125 ng/ml glial derived neurotropic factor, and 250 ng/ml fibroblast growth factor 1 (A): After 8 days of culture a UB was subdivided into thirds and re-cultured in new 3D ECM gels with fresh media (day 0). Over the course of next 8 days, these second-generation UB was subsequently subdivided into thirds and re-cultured within ECM gels for an additional 8 days. Growth and branching was evident, but to a lesser extent than that seen in the original subdivision. Scale bar=100 μm for all images. (B) A second--generation PB was subsequently subdivided into thirds and re-cultured within ECM gels for an additional 8 days. Growth and branching was evident, but to a lesser extent than that seen in the original subdivision. Scale bar =100 μm for all images. (C) A schematic representation of PE propagation demonstrates a potential colony with a large number of PBs derived from a single progenitor body, which can then be recombined with separately propagated mesenchyme.

FIG. 10 demonstrates a proposed in vitro kidney engineering strategy. A Wolffian duct bud (Na)) is first isolated and then is induced to bud. Each resulting bud can be isolated and further induced to undergo branching. The branched in vitro-formed ureteric bud (UB) is then recombined with metanephric mesenchyme (MM). After 4-6 days of mutual induction, the recombined tissue resembles a late-stage embryonic kidney. The recombined tissue is then implanted into a host animal where it is vascularized and forms glomeruli. The possibility of using cells to engineer ND and/or MM-like tissue is also indicated.

### DETAILED DESCRIPTION

As used herein and in the appended claims, the singular forms “a,” “and,” and “the” include plural referents unless the context clearly dictates otherwise. Thus, for example, reference to “a protein” includes a plurality of such proteins and reference to “the progenitor cell” includes reference to one or more progenitor cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Nearly all epithelial and endothelial organs are formed around branching ducts. While these ducts have organ-specific function, particularly with respect to the expression of plasma membrane channels and transporters, it is worth noting that much of the organ-specific function appears to be conferred by surrounding mesenchyme. For example, when branching ducts of the developing kidney are recombined with lung mesenchyme, surfactant proteins are made. In other words, when in vitro branched ureteric bud of the developing kidney are combined with mesenchyme, it is clear that the mesenchyme confers organ-specific gene expression (e.g., transporters, water channels), as well as structure and functionality characteristics of the maturing organ. Accordingly, it should be understood that the methods of the disclosure can be used to generate any number of desired proto-organs based upon the selection of the particular mesenchyme in combination with a source of budding tissue, such as intermediate mesoderm, the metanephric mesenchyme (MM), and the ureteric bud, Wolffian duct bud, or ureteric and Wolffian duct bud tissue (UB, WD, UB and WD).

A wide variety of tissue engineering strategies have being employed in attempts to “build” organ-like tissues. Due to the rapid development of Regenerative Medicine, many new/existing technologies such as cell sourcing, biocompatible scaffolds, bioreactors, 3D printing and other methods have been improved upon or developed. Accordingly, it is entirely conceivable that a complex organ like a failing kidney can be replaced with a tissue-engineered construct.

Branching morphogenesis is driven by a core program involving' many genes, resulting in cellular proliferation and migration such that “iterative tip-stalk generation” occurs. There appears to be a core program involving 100-200 genes that are involved in the process. Various branching morphogenesis associated genes have been identified by applying systems biology methods to “minimal” 3D in vitro models of branching ducts, and synthesized 3D in vitro and in vivo knockout data (resulting in branching defects). A number of growth factors necessary for branching have been identified by using these methods, including key matrix molecules and integrin. Furthermore, some of these have been verified in vivo using tissue-specific knockouts in breast and kidney. Accordingly, a core set of genes and factors associated with breast and kidney development have been identified and verified using in vitro and in vivo based methods (3D cell culture branching, 3D modified organ culture branching and in vivo knockout data). Examples of some these gene and factors are presented in TABLE I.

TABLE I

| Branching morphogenesis | Organ System | | |
|---|---|---|---|
| modulators | Kidney | Mammary gland | Lung |
| Growth factors and Receptors | | | |
| Fgf1 | X | | X |
| Fgf2 | X | X | |
| Fgf7 | X | X | |
| Fgf10 | X | X | X |
| Hgf | X | X | X |
| Tgfa | X | X | |
| Activin | X | X | |
| Bmp4 | X | X | X |
| Tgfβ | X | X | X |
| Gremlin1 | X | | X |
| ErbB/neuregulin/heregulin | X | X | |
| Fgfr2 | X | X | X |
| Egfr | X | X | |
| Transcription Factors | | | |
| Pax2 | X | X | |
| Eya1 | X | | X |
| Six1 | X | | X |
| Wnt4 | X | X | |
| Wnt5a | | X | X |
| Wnt11 | X | X | |
| B-Catenin | X | X | X |
| Pea3/Etv4 | X | X | X |
| Extracellular matrix | | | |
| Hs2st | X | X | |
| MMP2 | X | X | X |
| MT1-MMP | X | X | X |
| Fibronectin | X | X | X |
| Other Molecules | | | |
| Notch | X | X | X |
| Sonic Hedgehog | X | X | X |
| Sprouty1 | X | | X |
| Sprouty2 | | X | X |
| SFRP1 | X | | X |
| Semaphorin3a | X | | X |
| Slit/Robo | X | X | X |
| Eph/Ephrin | X | | X |

Central to epithelial organogenesis of the kidney, lung, breast, prostate, salivary gland, pancreas and other epithelial organs is the process of branching morphogenesis. It has been the subject of study for many years in the field of organogenesis—using such diverse approaches as in vivo gene knockouts, whole organ culture, partial organ culture in 3D (e.g., isolated UB culture in a 3D matrix), culture of epithelial cells in gels stimulated by various growth factors and other methods. Although there are differences between various epithelial organs, in general, the developmental process begins with the formation of an epithelial bud, which then undergoes repetitions of branching to create new tips and stalksoften in a stereotypical pattern (roughly 20 rounds in the developing human kidney, for example, and 24 in the developing lung).

Essentially the epithelial bud becomes an “iterative tip-stalk generator” (ITSG). This generator seems to be “powered” by a network involving dozens of genes which, despite differing in some specifics from organ to oran (i.e., the particular set of fibroblast growth factors, TGF-beta superfamily members, other heparin-binding growth factors, intracellular signaling pathways, integrin receptors, heparin sulfate proteoglycans involved, transcription factors) are highly reproducible when one looks at in vivo (i.e., knockout) and in vitro studies of each organ's development.

Cell culture models of branching morphogenesis using renal adult or embryonic cell lines (i.e., MDCK, IMCD and UB cells) indicate the importance of several growth factor signaling pathways (including those mediated epidermal growth factor receptor-ligands, hepatocyte growth factor, various TGF betas and BMBs, pleiotropin, various FGFs and others) in regulating branching morphogenesis in 3D extracellular matrix gels (e.g., see FIG. 2). Many of the ECM. proteins important for branching, their integrin receptors, heparin-sulfate proteoglycans, intracellular signaling pathways and ECM-digesting proteases MMPs) have been identified. The isolated ureteric bud (the embryonic primordial tissue out of which the branched urinary collecting duct system of the kidney arises) undergoes branching morphogenesis using many of the same molecules as the epithelial cells in 3D culture (or closely related molecules). Initially, the murine knockout data in the kidney development field suggested that some of these molecules might be less important in vivo than in vitro. But it is now becoming evident, from more careful examination of the knockouts, as well as from double knockouts, that similar sets of: genes are important for branching of the ureteric bud in vivo. For instance, the hepatocyte growth factor (c-met receptor) was first directly implicated in branching of MDCK cells. However, in other renal epithelial cell lines (UB and IMCD cells), arguably more relevant. to UB and collecting duct development, EGF receptor ligands appeared equally important. Nevertheless, organ culture studies clearly suggest that HGF is important for branching. It was subsequently shown that, whereas the single knockout of the HGF-receptor (c-met) does not result in a detectable phenotype, the double knockout of c-met and the EGF receptor has a branching defect, which was predicted from in vitro UB and IMCD cell culture studies. Furthermore, in mammary gland, knockout of c-met alone alters branching. This is but one example of the growing concordance of in vitro and in vivo data on branching morphogenesis. Although there is some variability from organ to organ, similar sets of genes are implicated in epithelial branching in a number of organs. The similarities between the kidney, the lung, the mammary gland, prostate and salivary gland are particularly striking. With the significant overlap between the genes and pathways leading to organ development, it is reasonable to assume that there is a “universal” epithelial “iterative tip-stalk generator” or ITSG, powered by a branching gene network, around which epithelial organs may be built.

In creating the innovative in vitro models disclosed herein a relatively small number of relatively homogeneous cells (art embryonic bud) when presented with the correct set of growth factors, can in the appropriate 3D matrix, be differentiated into a “tip-stalk generator” that is capable of continuous ductal branching. The tip-stalk generator is only limited by “bioreactor” considerations such as nutrition, oxygenation and mixing. Indeed, these tips can be subcultured into branching structures in a culture system much as a gardener might propagate cuttings from a “parent” tree—and once these grow into branching structures, their tips cart be subcultured again and again. Thus, from a small number of cells induced to undergo in vitro branching, hundreds if not thousands of independent branching ductal systems can be generated by taking advantage of the “tip-stalk generator” mechanism which is empowered by branching morphogenesis driven by growth factors and core gene program.

Most epithelial and endothelial organs are built around or from these propagatable 3D iterative tip-stalk generators. Examples of organs include but are not limited to, adrenal glands, appendix, gall bladder, kidney, liver, lung, pancreas, parathytoid. gland, pineal gland, pituitary gland, spleen, thymus, thyroid gland, and vermiform appendix. As mentioned above, the mesenchyme appears more crucial in providing, through cell contact and short-acting factors, organ-specific features. Thus, the 3D branching ductal structure can be viewed as a “cellular scaffold” which can secrete a relatively insoluble “pro-branching matrix scaffold” that can be as important as the cellular scaffold (e.g., a “cellular and matrix” scaffold).

In the presence of a particular growth factor and matrix-related molecule “cocktail” (comprising of soluble factors derived from the MM), the progenitor cells become an impressive iterative tip-stalk generator in vitro. The growth factor and matrix--related molecule “cocktail”, is very similar to the sets of molecules thought to be important for branching morphogenesis in the developing mammary gland, lung and other epithelial organs. This is all, the more remarkable because of the very distinct roles that these organs, when fully differentiated, play in systemic physiology; consider, for instance, the different physiological functions of the kidney, lung and mammary gland. It is also interesting because, while most are cases of predominantly prenatal development through branching (e.g., kidney, lung, pancreas), others (e.g., mammary gland, prostate) are cases of predominantly postnatal development.

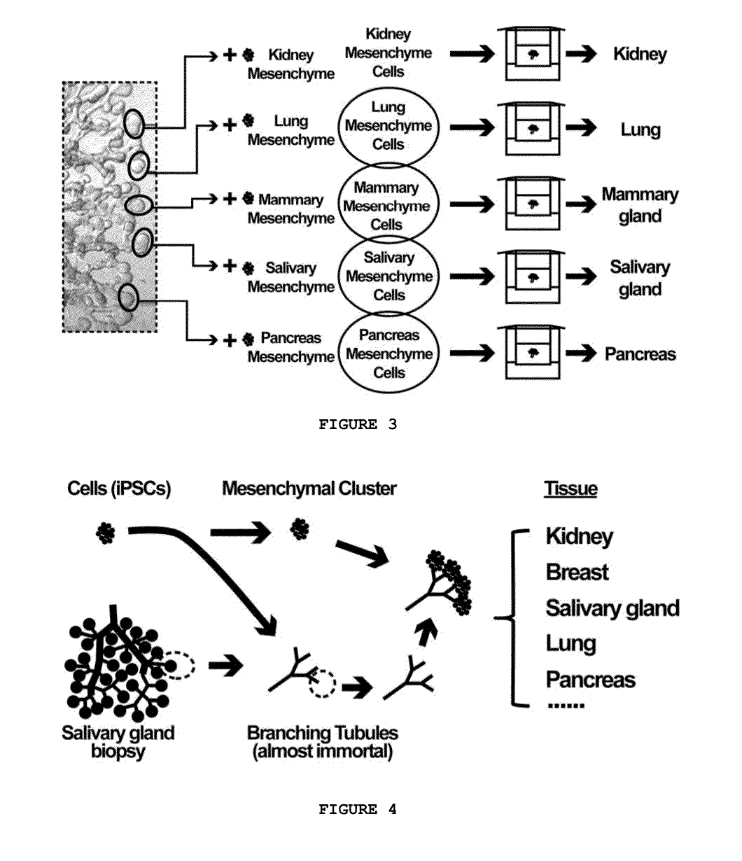

Unlike the kidney, where the mesenchyme (MM) actually becomes part of the structural and functional epithelial unit (the nephron) after transforming into epithelia via a mesenchymal-to-epithelial transition (MET), the mesenchyme of other developing organs do not appreciably epithelialize and become incorporated into the functional ductal epithelial units of epithelial organs, such as the mammary gland and lung. The mesenchyme, however, plays a key instructive and generalized role across most if not all developing organs (e.g., see FIG. 3).

Of particular interest in this regard are recombination experiments between branching epithelial structures of various organs with the mesenchyme of a different organ. When the branching ureteric bud (UB) of the developing kidney is recombined in vitro with lung mesenchyme, the kidney-derived branching epithelial cells produce surfactant protein. Likewise, when the kidney mesenchyme (MM) is recombined with the branching lung bud, one lung derived epithelial tissue expresses kidney water channel (aquaporin) protein.

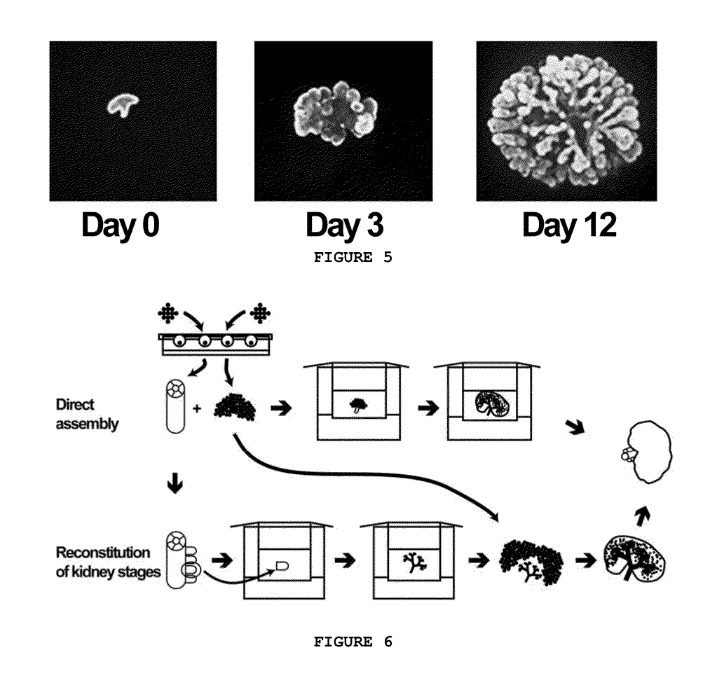

The “universal ITSG”, therefore, has the potential to be recombined with organ-specific mesenchyme, or mesenchymal cells differentiated towards that organ, to build whole organs of a type which would be determined by the kind of mesenchyme used in the recombination (e.g., see FIG. 4). Importantly, this “universal ITSG” forms a three dimensional branching tree that will serve as a “3D cellular scaffold” for the engineered tissue even as it differentiates and acquires organ-specific characteristics (e.g., see FIG. 5).

As various sources of stem cells, progenitor cells, and mesenchymal cells have been differentiated down to particular organ-specific cell lineages, large numbers of organ-specific mesenchymal cells are readily available. Further, if these organ-specific cell lineages are derived from iPSCs, these organ-specific mesenchyme cells could also be patient-specific (e.g., see FIG. 4). In a particular embodiment, mesenchyme differentiation can be achieved by using the protocols and methods presented in Martovetsky at al. (Mol Pharm 84:1-16 (2013)), which is incorporated herein in-full.

Metaphorically one can visualize a ‘gardener's strategy’ to generate a large number of 3D tip stalk generators from a small number of cells. One might imagine taking clippings of branches and planting them separately. In other words, one could micro-dissect and subculture tips (or tips and stalks) in new 3D matrices with the appropriate growth factors and supplements (within some sort of bioreactor). And when these ITSGs branch sufficiently, the process can be repeated. Thus, by taking advantage of the intrinsic “power” of the branching process (presumably this power derives from the network of interacting genes responsible for branching morphogenesis) a single ITSG serves as abundant source of other ITSGs.

In other words, by using the ‘gardener's strategy’ outlined above, a 3D tip-stalk generator can be differentiated from a small number of cells (tip-like cells or tip itself), which can then through branching' morphogenesis produce a huge number of independent branching ductal “cellular scaffolds (with pro-branching matrix).” Out of which, an independent organ can arise from each one of these, when interfaced with the appropriate organ-specific mesenchyme. It is akin to putting apples back on a barren tree to create one type of organ (e.g., kidney), putting lemons back on the same type of tree (propagated from a cutting of the same mother tree) to create another type of organ (e.g., lung), and Putting oranges back on a similar barren tree to create a third organ (e.g., pancreas) with the possibility that one can also make a tree that produces two different fruits (e.g., lemon/lime trees) or a hybrid fruit (e.g., tangelo).

As disclosed herein, experiments have been performed which validate this ‘gardener's strategy’ using isolated UB cultures in the context of developing kidneys. When grown sufficiently in 3D culture, portions containing tips and stalks were microdissected and then recultured. The recultured isolated UBs, when grown into larger branched structures, were then recombined with kidney mesenchyme (MM) to yield kidney organoids that appeared similar to the embryonic kidney in whole organ culture. The MM-derived nephron connected to the UB derived branching collecting ducts. This process was repeated through another generation. A single UB therefore yielded many new UB-like structures, and when these new UB-like structures were recombined with MM, they became kidney organoids reminiscent of the embryonic kidney itself with, at least superficially, the appropriate tubular plumbing set up (i.e., connections between the distal tubule and the collecting duct). Cells capable of forming a microvasculature can be induced to branch alongside the 3D cellular scaffold of the ITSG in an in vitro culture system such as the isolated ureteric bud. Overlapping or similar sets of growth factors and extracellular matrix molecules play key roles in branching of epithelial cells and endothelial cells.

The methods disclosed herein, provide for endothelial cells generated from embryonic stem cells that are functional, transplantable and responsive to microenvironmental signals. The embryonic-derived endothelial cells of the disclosure are versatile, so they can be transplanted into different tissues, become educated by the tissue, and acquire the characteristics of native endothelial cells.

In a particular embodiment, the disclosure provides for endothelial cells that can be derived from human embryonic stem cells (hESCs) as well as by somatic cell nuclear transfer (SCNT). In the SCNT approach, the nucleus of a somatic cell is introduced into the human egg resulting in the generation of embryonic stem cells that would generate endothelial cells that are a genetic match of the patient. Furthermore, one can take cells discarded after a diagnostic prenatal amniocentesis and turn them into endothelial cells capable of repairing and regenerating blood vessels. Freezing and stockpiling such cells will allow transplantation of these cells to a genetically diverse population of patients.

Endothelial differentiation of hESCs cart be induced by two methods: 2D culturing of the cells on extracellular matrix (ECM) or a feeder layer, which can induce directed differentiation toward endothelial lineages, or growing hESCs in a 3D system in a differentiation medium to form embryoid bodies (EBs), which induce spontaneous differentiation into the various cell types of the three germ layers. Endothelial cells can then be isolated for further differentiation and maturation. Vascular endothelial growth factor receptor-2 (VEGFR2) , CD133, CD31 and CD34 are expressed in endothelial and hematopoietic progenitor cells in relating human tissues, whereas VEGFR2 and CD133 are also expressed in undifferentiated hESCs. With respect that CD31 and CD34 are not expressed (or minimally expressed) in undifferentiated hESCs, they are chosen for isolating hESC-derived endothelial progenitor cells (EPCs). Isolated CD34 cells can be cultured in endothelial growth media containing VEGF and basic fibroblast growth factor (hFGF) for 7-10 days. The hESC-derived endothelial cells are characterized by specific endothelial markers, including CD31, vascular/endothelial (VE)-Cadherin, von Willebrand factor (vWF), VEGFR2 (KDR), and Tie-2. These cells are capable of uptaking DiI-acetylated low-density lipoprotein (DiIi-Ac-LDL) and form vascular network-like structures when placed on Matrigel. Accordingly, the disclosure provides for the production of vascular network-like structures comprising contacting a stem cell or branching endothelial cell with a cell survival agent or biological active agent to stimulate growth and proliferation; contacting the cells with a branching agent that promotes formation of tubular tissue branches and/or globular morphology to generate budding tissue; combining the bud tissue with tissue specific mesenchyme in a biocompatible matrix; and culturing the combination to form vascular network-like structures in vitro.

While most of branching morphogenesis occurs during embryonic development of organs, in some cases, such as the mammary and prostate glands, it can occur after birth. If, for the purpose of obtaining a patient-specific ITSG, it is important to begin with ductal cells that have the ability to branch after birth, these are relatively accessible tissues. But it may be possible to use mature cells; some of the animal cell lines that have been quite useful in studying branching morphogenesis (e.g., IMCD cells) are derived from adult organs. Accordingly, one can generate a patient-specific ITSG, for example, from an adult salivary gland biopsy. Further, patient-derived IPSCs can be used as a starting point for the ITSG.

The disclosure provides for a “universal” epithelial ITSG which has sufficient flexibility to differentiate into the branched ducts or tubules of organs as functionally distinct as the kidney, lung, pancreas, salivary gland, breast, prostate, thyroid, and biliary tract. These distinct functions arise, among other things, as a result of the appropriate express non of tissue-specific sets of transporters and channels at the apical and basolateral surfaces of the polarized epithelial cells lining the ducts and tubules of different epithelial organs. For example, the proximal tubule of the kidney must be capable of vectorial transport (usually plasma to urine) of drugs and toxins, and the collecting ducts must be capable of concentrating the urine by absorbing large amounts of water. The regulation of these and other tissue-specific genes are governed by particular sets of transcription factors that can be activated in the proper spatio-temporal contexts. While the organ-specific mesenchyme cells disclosed herein are capable to induce the ITSG to adopt the functional tissue-specific properties of mature ducts and tubules, it should be understood that the disclosure further provides for cell sources, matrices, dimensionality, bioreactors and exogenous agents (e.g., small molecules, growth factors) that further facilitate the differentiation of the ITSG towards one organ or another.

The disclosure provides for engineered organ-like constructs that are useful for tissue toxicity studies, developmental biology and physiology that help minimize the need for in vivo animal studies. The disclosure also provides for engineered organ-like constructs that are suitable for incorporation into medical device, for transplantation or for other regenerative medicine purposes. Going back to the example of the kidney, renal insufficiency is frequently associated with chronic systemic diseases (e.g., diabetes, hypertension, systemic lupus erythematosis), and it can therefore be expected that the engineered organ-like constructs can be used to treat or ameliorate the symptoms of countless diseases, conditions or disorders. In yet a further embodiment, the disease, disorder or condition is selected from the group consisting of Sjogren syndrome, Addison's disease, Celiac disease, chronic thyroiditis, multiple sclerosis, systemic lupus erythematosus, diabetes, pancreatitis, hypertension, chronic kidney disease, polycystic kidney disease, end stage renal disease, malignant hypertension, acute liver failure, chronic liver failure, chronic hepatitis infection, liver cirrhosis, hemochromatosis, Wilson's disease, nonalcoholic steatohepatitis, hepatocellular carcinoma, hepatoblastoma, cholangiocarcinoma, biliary artesia, coronary artery disease, cardiomyopathy, heart failure, cystic fibrosis, emphysema, obstructive lung disease, short bowel syndrome, necrotizing enterocolitis, and Crohn's disease. In another embodiment, the disclosure provides a method to reconstruct tissue that has been removed from a subject comprising: implanting a tissue or a portion of the tissue made by the methods described herein in a subject to replace or reconstruct damaged tissue (e.g., damage resulting from diseases, age-related damage or natural deterioration of tissue due to age, damage resulting from accidents, and damage resulting from the environment). In a particular embodiment, breast tissue created by the methods disclosed. herein can be used to replace or reconstruct breast tissue which has been removed via a mastectomy.

The disclosure provides for a “generic” 3D branching ductal “cellular scaffold” which can be used for making different branching organs. The “organ type” (e.g., lung or kidney) is created by simply recombining with the appropriate mesenchyme cells (e.g., organ-specific mesenchymal cell type). One can imagine, following the protocol outlined above, having a renewable source of branching ductal systems and recombining at will with the particular (e.g., lung, kidney, salivary gland) mesenchyme cells, possibly from patient-derived cells (see below) to create each organ whenever one needs to do so.

In a particular embodiment, the disclosure provides that the source of mesenchyme is early embryonic kidney (metanephric) mesenchyme. In an alternate embodiment, the source of mesenchyme is iPS cells, adipose cells, or other type mesenchymal stem cells which are capable of being differentiated to organ-specific mesenchyme. In a further embodiment, the source of mesenchyme is embryonic fibroblasts which are differentiated to organ-specific mesenchyme. In yet a further embodiment, the source of mesenchyme is cells derived from bone marrow which are differentiated to organ-specific mesenchyme. In another embodiment, the source of mesenchyme is early embryonic kidney (metanephric) mesenchyme which is differentiated to organ-specific mesenchyme.

Through Chip-seq analysis of enhancer regions and transcriptional start sites (during metanephric mesenchyme differentiation into drug-transporting proximal kidney tubule,) key sets of transcription factors capable of facilitating differentiation to organ specific mesenchyme can be identified. However, it should be understood that the disclosure and the methods described herein can be used for many or most type of organs. The experiments of the disclosure thereof provide a proof of concept that the strategies disclosed herein are effective, as exemplified by the successful production of kidney tissue. The methods disclosed herein, however, enable the production of any epithelial and/or endothelial organ. In a particular embodiment, the organ specific mesenchyme is pancreatic mesenchyme. In a certain embodiment, the organ specific mesenchyme is breast mesenchyme. In another embodiment, the organ specific mesenchyme is lung mesenchyme. In yet another embodiment, the organ specific mesenchyme is gall bladder mesenchyme. In a further embodiment, the organ specific mesenchyme is spleen mesenchyme. In yet a further embodiment, the organ specific mesenchyme is liver mesenchyme. In a particular embodiment, the organ specific mesenchyme is glandular mesenchyme tissue, including adrenal, salivary, prostate, thymus, parathyroid, pituitary, and pineal mesenchyme.

For the branching ductal cellular scaffold, one can start with an embryonic bud itself (e.g., lung, kidney, and breast), epithelial buds constructed from reprogrammed stem cells and the like. For example, the branching ductal cellular scaffold can originate from a biopsy containing epithelial and/or endothelial cells from a subject. It should therefore be understood that virtually any type of endothelial or endothelial cell (e.g., salivary gland, mammary, liver, prostate or blood vessel) can be differentiated into a branching “universal ITSG,” as disclosed herein.

Indeed, any epithelial organ tip will do so long as it has an “active branching gene network.” One can create a “generic” branching ductal system from easily accessible branching epithelial tissue such as pubertal breast, pregnant breast or prostate. If branch tips from mature tissue are used they will branch again in 3D culture with the right growth factor-small molecule-matrix milieu etc. So, following the logic above, it is possible to make a lung from a salivary gland biopsy plus a lung-specific mesenchyme cell that has been reprogrammed from a stem cell that is being maintained in a “poised state” in culture.

There is also the possibility of creating multi-functional branched organs utilizing the strategies disclosed herein. For example, one can engineer something that can function as either a kidney or pancreas when implanted in the right environment, or one that can function as both. This would be of obvious value in, say, a diabetic with chronic kidney disease, a very common scenario.

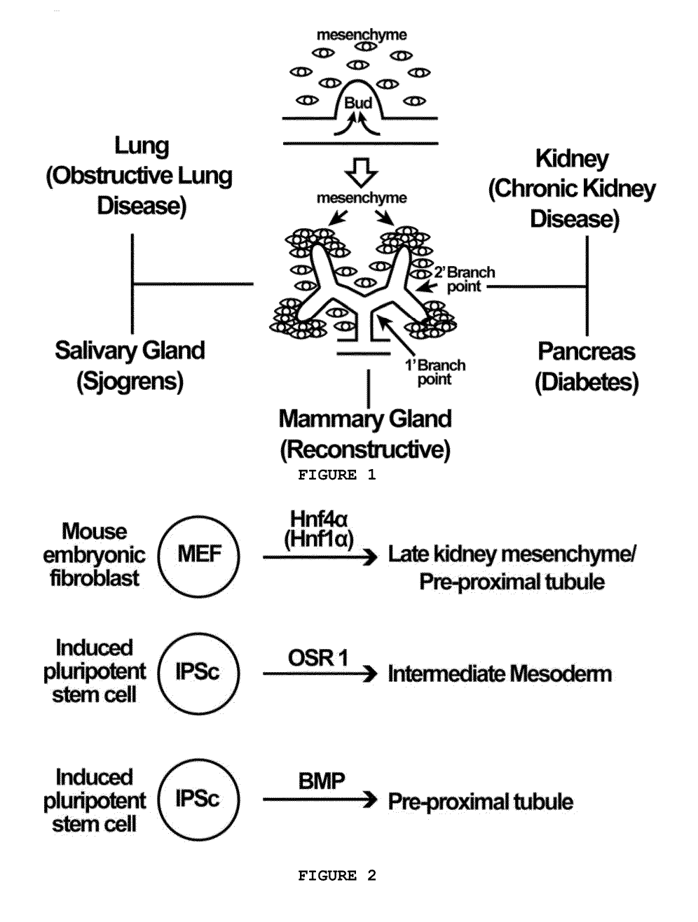

By using approaches similar to those described herein, one can culture and propagate vascular trees capable of providing vasculature for engineered tissues and organoids or for providing vasculature for independent medical needs (e.g., diabetes, atherosclerosis or other chronic diseases where the tissue blood vessel supply is compromised) or surgical needs (e.g., organ transplantation, trauma or other vascular surgery settings) (e.g., see FIG. 1).

Apart from soluble growth factors similar to those involved in epithelial or endothelial branching (including HGF, various FGFs, EGF receptor ligands, BMPs, activins and others), VEGF and/or PDGF are likely to be required as part of the growth factor cocktail as well as morphogenetic small molecules. For organ specific type vascular growth and differentiation, contact or proximity with mesenchymal cells, derived from, e.g., a stem cell (iPS, cord, bone marrow, placental) or other source may be required. The strategy is similar to that described for branched epithelial organs.

The disclosure provides for a set of organ specific mesenchyme cells guiding 3D epithelial branching to produce organ tissue. While the disclosure further provides for vascular supply development for the organ tissue. In a particular embodiment, the organ tissue production and vascular supply development are performed in different bioreactor steps. In an alternate embodiment, the organ tissue production and vascular supply development are performed in the same bioreactor step. The epithelial and vascular network-like trees would be derived from the propagated 3D branched epithelial and endothelial trees. The organ-specific mesenchyme would be derived from, e.g., a stem cell-like source. The end result would be a vascularized organoid and, depending on the organ-specificity of the mesenchyme, many different vascularized organoids can be made. In a particular embodiment, the vascularized organ or organoid tissue produced is breast tissue. In a certain embodiment, the vascularized organ or organoid tissue produced is pancreatic tissue. In another embodiment, the vascularized organ or organoid tissue produced is lung tissue. In yet another embodiment, the vascularized organ or organoid tissue produced is gall bladder tissue. In a further embodiment, the vascularized organ or organoid tissue produced is spleen tissue. In vet a further embodiment, the vascularized organ or organoid tissue produced is liver tissue. In a particular embodiment, the vascularized organ or organoid tissue produced is reproductive tissue. In a certain embodiment, the vascularized organ or organoid tissue produced is glandular tissue, including adrenal, salivary, prostate, thymus, parathyroid, pituitary, and pineal tissue. In another embodiment, the vascularized organ or organoid tissue produced is vascular tissue.

The disclosure provides a cell-based epithelial and/or vascular network development strategy. Unlike prior strategies, which have used tissue segments and recombination, the present disclosure demonstrates the ability to develop tissue components from substantially homogenous cell types followed by recombination of the tissue components. In a particular embodiment, stem cells are used as the initial cell type source for the methods disclosed herein.

The term “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew its line or to produce progeny cells which will differentiate into a desired cell type; or a lineage-committed progenitor cell and its progeny, which is capable of self-renewal and is capable of differentiating into a further lineage defined cell type. Unlike pluripotent stem cells, lineage-committed progenitor cells are generally considered to be incapable of giving rise to numerous cell types that phenotypically differ front each other, instead, they give rise to one or possibly two lineage-committed cell types. As a further description, stem cells are cells capable of differentiation into other cell types, including those having a particular, specialized function (e.g., tissue specific cells, parenchymal cells and progenitors thereof). Progenitor cells (i.e., “multipotent”) are cells that can give rise to different terminally differentiated cell types, and cells that are capable of giving rise to various progenitor cells. Cells that give rise to some or many, but not all, of the cell types of an organism are often termed “pluripotent” stem cells, which are able to differentiate into any cell type in the body of a mature organism, although without reprogramming they are unable to de-differentiate into the cells from which they were derived. As will be appreciated, “multipotent” stem/progenitor cells have a more narrow differentiation potential than do pluripotent stem cells. Another class of cells even more primitive (i.e., uncommitted to a particular differentiation fate) than pluripotent stem cells are the so-called “totipotent” stem cells (e.g., fertilized oocytes, cells of embryos at the two and four cell stages of development), which have the ability to differentiate into any type of cell of the particular species. For example, a single totipotent stem cell could give rise to a complete animal, as well as to any of the myriad of cell types found in the particular species (e.g., humans).

Embryonic stem cells are generated and maintained using methods well known to those of skill in the relevant art, such as those described by Doetschman et al. (J. Embryol. Exp. Mol. Biol. 87:27-45 (1985)). Any line of ES cells can be used. One mouse strain that is typically used for production of ES cells is the 129J strain. Another ES cell line is murine cell line D3 (American Type Culture Collection, catalog no. CKL 1934). Still another ES cell line is the 896 cell line. Human embryonic stem cells (hESCs) can be isolated, for example, from human blastocysts obtained from human in vivo preimplantation embryos, in vitro fertilized embryos, or one-cell human embryos expanded to the blastocyst stage as described in Bongso et al. (Hum. Reprod. 4:706 (1989)). Human embryos can be cultured to the blastocyst stage in G1.2 and G2.2 medium as described in Gardner et al. (Fertil. Steril. 69:84 (1998)). The zona pellucida is removed from blastocysts by brief exposure to pronase (Sigma). The inner cell masses can be isolated by immunosurgery or by mechanical separation, and are plated on mouse embryonic feeder layers, or in the defined culture system as described herein. After nine to fifteen days, inner cell mass-derived outgrowths are dissociated into clumps either by exposure to calcium and magnesium-free phosphate-buffered saline (PBS) with 1 mM EDTA, by exposure to dispase, collagenase, or trypsin, or by mechanical dissociation with a micropipette. The dissociated cells are then re-plated as before in fresh medium and observed for colony formation. Colonies demonstrating undifferentiated morphology are individually selected by micropipette, mechanically dissociated into clumps, and re-plated. Embryonic stem cell-like morphology is characterized as compact colonies with apparently high nucleus to cytoplasm ratio and prominent nucleoli. Resulting embryonic stem cells are then routinely split every 1-2 weeks by brief trypsinization, exposure to Dulbecco's PBS (without calcium or magnesium and with 2 mM EDTA), exposure to type IV collagenase (about 200 U/mL) , or by selection of individual colonies by mechanical dissociation, for example, by using a micropipette.

Once isolated, the stem cells can be cultured in a culture medium as described herein which supports the substantially undifferentiated growth of stem cells by using any suitable cell culturing technique. For example, a matrix layer can be provided prior to lysis of primate feeder cells (preferably allogeneic feeder cells) or a synthetic or purified matrix can be prepared using standard methods. The stem cells to be cultured are then added atop the matrix along with the culture medium, in other embodiments, once isolated, undifferentiated stem cells can be directly added to an extracellular matrix that contains laminin or a growth-arrested human feeder cell layer (e.g., a human foreskin fibroblast cell layer) and maintained in a serum-free growth environment, according to the culture methods of invention. In yet another embodiment, the stem cells can be directly added to a biocompatible cell culture plate in the absence of an extracellular matrix material (e.g., directly on polystyrene, glass or the like). Unlike existing embryonic stern cell lines cultured using conventional techniques, embryonic stem cells and their derivatives prepared and cultured in accordance with the methods of the disclosure avoid or have reduced exposure to xenogeneic antigens that may be present in feeder lavers. This is due in part to the media compositions promoting growth in the absence of feeder layers or directly on a cell, culture substrate. This avoids the risks of contaminating human cells, for example, with non-human animal cells, transmitting pathogens from non-human animal cells to human cells, forming heterogeneous fusion cells, and exposing human cells to toxic xenogeneic factors.

Endothelial cells, the cells that make up the structure of blood vessels, drive regeneration in organ tissues by releasing beneficial, organ-specific molecules. Organs dictate the structure and function of their own blood vessels, including the repair molecules they secrete. Each organ is endowed with blood vessels with unique shape and function and delegated with the difficult task of complying with the metabolic demands of that organ. Endothelial cells possess tissue-specific genes that code for unique growth factors, adhesion molecules, and factors regulating metabolism. The endothelial cells of the disclosure are derived from embryonic stem cells and behave as resilient endothelial cells, being able to be taught how to act like an organ-specific blood vessel.

The stem cells can be directed to form branching buds or a particular mesenchymal cell type. For example, co-culturing the stem cells with epithelial bud cells and/or in the presence of conditioned media, pleotrophin, GDNF etc. can cause the stem cells to differentiate to a particular epithelial budding cell type. Alternatively, mesenchymal cells can be derived from the stem cells by co-culturing the stem cells with a particular mesnechymal stem cell or in the presence of conditioned media derived from a mesenchymal stem cell culture.

In yet another aspect, mesenchymal stem cells (MSCs) are used. Mesenchymal stem cells are multipotent stem cells. Mesenchyme is embryonic connective tissue that is derived from the mesoderm and that differentiates into hematopoietic and connective tissue. MSCs can be obtained from both marrow and non-marrow tissues, such as adult muscle side-population cells or the Wharton's jelly present in the umbilical cord. These mesenchymal stem cells provide an excellent source of mescenchymal cells used in the methods of the disclosure.

Substantially homogenous populations of cells (e.g., 80, 90, 95, 98, 99 or 100% homogenous) can be used in the development of mesechnyme tissue or ureteric bud and Wolffian duct cells. For example, many epithelial and endothelial organs, such as kidney, lung, and prostate under go branching morphogenesis in the course of development. The kidney is formed by mutual induction between two tissues derived from the intermediate mesoderm, the metanephric mesenchyme (M, and the ureteric bud, Wolffian duct bud, or ureteric and Wolffian duct bud (UB, WD, UB and WD). The UB, ND, or UB and WB induces the MM to differentiate and form one proximal nephron, while the UB, ND, or UB and ND undergoes dichotomous branching and elongation as it invades the MM, ultimately forming the kidney collecting system (e.g., see FIG. 6).

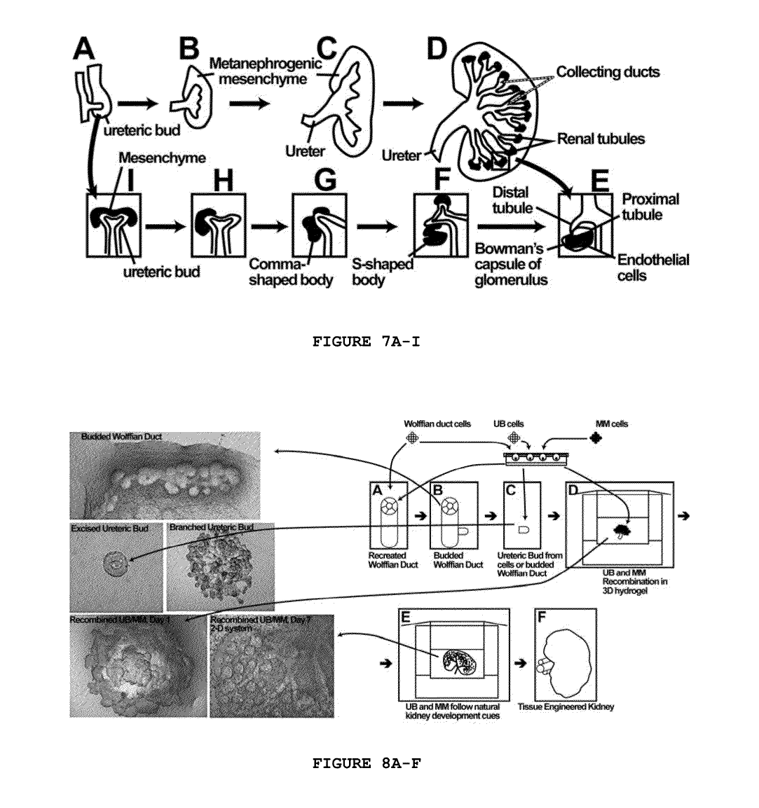

Because of the many different morphogenetic processes and cell types involved, the kidney is one of the most complex developing epithelial organs. The metanephric kidney begins as an epithelial bud, known as the ureteric bud (UB), which is an outgrowth of the Wolffian duct (e.g., see FIG. 7A-I). This process occurs at about embryonic day 12 in the rat (and around week 4 in the human fetus). Under the influence of soluble factors and cell-cell interactions with the metanephric mesenchyme (MM), the newly formed UB undergoes iterative tip-stalk generation to form the developing collecting duct system—a differentiated tree emerging from multiple rounds of UB branching. This collecting duct tree, ultimately consisting of around 1 million ductal tips in humans (formed from roughly 20 iterations of branching morphogenesis of the UB) feeds into the ureter. Meanwhile—in a process possibly unique to the kidney mesenchyme—the MM epithelializes and then undergoes tubulogenesis to form the more proximal parts of the nephron (the functional unit of the kidney), including the epithelial portion of the glomerulus (the filtration unit of the nephron), the proximal convoluted tubule, loop of Henle and distal tubule. These structures (formed from the MM) are involved in drug or toxin handling, regulation of salt balance and maintenance of acid-base homeostasis, whereas the collecting ducts (formed from UB branching) are responsible for water balance. Importantly, kidney development seems to occur in stages that can be defined morphologically, by distinct gene expression signatures or by computational analysis of global gene expression patterns. This is important in that there are, apart from functionality, several different measures of differentiation that could be applied to an organ-like structure produced by tissue engineering methods.

Much of (avascular) kidney development can be reproduced in organ culture of the embryonic kidney. Furthermore, the individual Processes of UB and MM morphogenesis have been, in large part, separated in vitro (ex vivo) using partial or modified organ cultures. Key soluble factors were initially identified using these and other in vitro models and then confirmed in vivo in knockout animals. Many components and integrin receptors have been identified and are provided for herein.

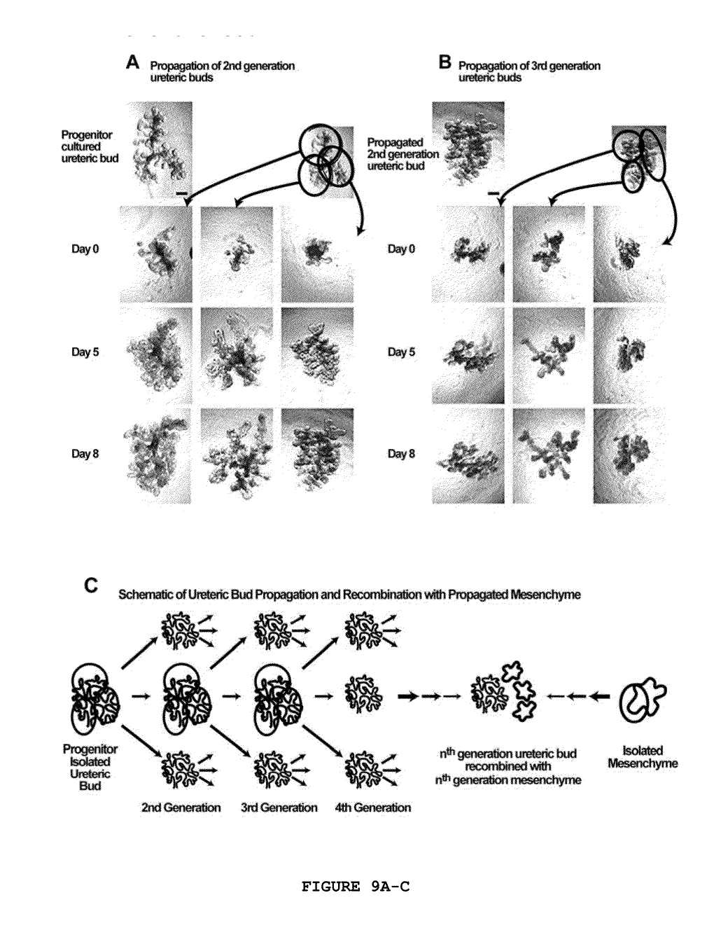

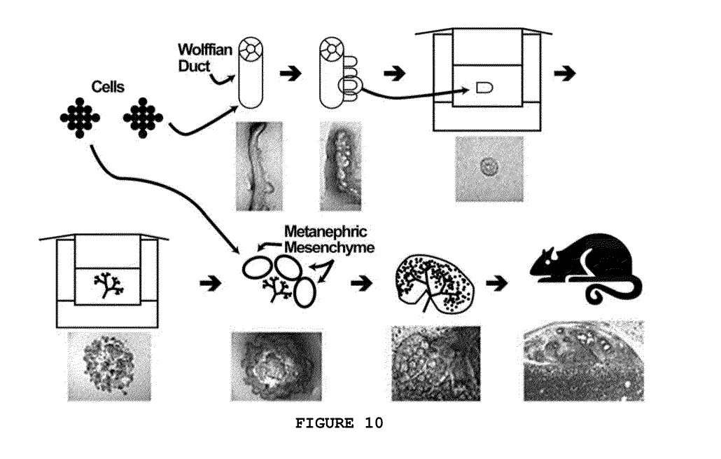

By Following a developmental logic, the kidney has been reconstituted into functional tissue from these partial organ cultures (FIG. 8A-F) ; this tissue is capable, for instance, of differentiated transport activity. By microarrays, these “engineered” kidneys have gene expression patterns similar to a E17-18 rat kidney (gestation being 22 days). It is known that the whole embryonic kidney can be transplanted into rodents and can develop some function. When the “engineered” kidney was transplanted into rodents, vascularized nephrons were observed. Related to this approach, cell-based methods have had some success. Essentially, these studies support the view that one can begin with cells instead of primordial tissues (e.g., see FIG. 10). Moreover, it is possible to dissociate the embryonic kidney into its cells, minimize apoptosis through rho-kinase inhibition, and the cells can reassemble into 3D kidney-like structures.

A number of factors are known to cause differentiation of stem cells or progenitor cells along a directed lineage specific for various tissues. Non-limiting examples of bioactive molecules include activin A, adrenomedullin, aFGF, ALK1, ALK5, ANF, angiogenin, angiopoletin-1, angiopoietin-2, angiopoietin-3, angiopoietin-4, angiostatin, angiotropin, angiotensin-2, At120-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-α, TGF-β, PD-ECGF, PDGF, IGF, IL8, growth hormone, fibrin fragment E, FGF-5, fibronectin, fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-.beta., PD-ECGF, PD-ECGF, PAI-2, PD-ECGF, PF4, P1GF, PKR1, PKR2, PPARγ, PPARγ ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), and nicotinic amide.

Soluble factors that have been thought to play a role in morphogenetic capacity include hepatocytes growth factor (HGF) and epidermal growth factor (EGF) receptor ligands, which have been shown to induce branching tubular structures in epithelial cells cultured in collagen gels.

The cells used in the generation of the engineered tissue as described herein can be induced to proliferate and/or differentiate using any number or combination of factors such as those described, above. In one aspect, the disclosure provides for culturing a stem cell in a culture medium comprising a bioactive molecule to expand the stem cells and/or cause differentiation of the stem cell into a branching bud. Such culture conditions can generate a population of bud cells that can be used as a component for generation of a functioning branching tissue. In another aspect, the same type of stem cell or a different stem cell type can be cultured in a culture medium comprising a bioactive molecule to expand the stem cells and/or cause differentiation of the stem cell into a mesenchymal cell. Such culture conditions can generate a population of mesenchymal cells that upon combination with branching bud cells can be used as a component for generation of a functioning branched tissue, the type of tissue dependent upon the phenotypic lineage of the meschenymal cells.

Hepatocyte growth factor (HGF), for example, has been shown to induce the formation of branching tubular structures with lumens in three-dimensional cultures of epithelial cell lines derived from adult kidneys (i.e., MDCK and mIMCD cells).

Another group of soluble factors implicated in branching morphogenesis of epithelial cells are the family of epidermal growth factor (EGF) receptor ligands. EGF receptor ligands are capable of inducing the formation of branching tubular structures with lumens in three-dimensional, cultures of mIMCD cells, a kidney cell line derived from adult collecting duct cells as described in Barros et al., 1995; and Sakurai et al., 1997.

Tubulogenesis is a phenotypic transformation of the cells such that condensed aggregates of tubule cells form about a central lumen wherein the lumen is bordered by cells possessing a polarized epithelial phenotype and tight junction complexes along the luminal border. For example, it has been shown that conditioned medium elaborated by MM-derived cell lines (BSN-CM) UBs, WDs, or UBs and WDs in three-dimensional culture to form branching tubular structures with clearly distinguishable lumens. Furthermore, GDNF plays a role in branching morphogenesis of isolated UB, WD, or UB and WD and cart be used with stem cells.

Studies in the developing mammalian lung and Drosophila trachea indicate that members of the FGF family function in branching morphogenesis of epithelial tissues. Furthermore, null mutations of either fgf7 or fgf10 have also been reported to affect kidney development, although in both cases the kidneys appear to be modestly affected. For example, in fgf7-null kidneys, there is a 30% reduction in the number of nephrons, and the kidneys appear to function normally. Moreover, since FGF7 is detected in the developing kidney after several iterations of UB, WD, or UB and WD branching has already occurred, it is likely that other factors are necessary for the early steps of the branching program. In the case of FGF 10, the defect appears similar. Nevertheless, by potentiating the effect of an essential branching morphogen produced by the MM, certain FEFs are demonstrated herein to play a role in stem cell, UP, ND, or UB and WD branching morphogenesis.

To further understand the disclosure, a branched kidney tissue will be described. As used herein, the abbreviation UB, WD, or UB and WD includes ureteric bud, Wolffian duct bud, or ureteric and Wolffian duct bud cells obtained from UB, WD, or UB and WD tissue, as well as UB, ND, or UP and ND tissue fragments, whole UB, WD, or UB and WD tissue, and UB, WD, or UB and WD cell lines, unless clearly indicated, otherwise in the specification. The UB, WD, or UB and WD cells may be primary cells obtained from embryonic kidney tissue by various techniques known in the art. Such primary UB, WD, or UB and WD cells are not immortalized (e.g., by SV40), but may be transfected and/or transformed to express a desired product, as discussed in more detail herein.

The culture system and methods of the disclosure provide the ability to propagate the isolated UB, WD, or UB and WD in vitro through several generations. For example, isolated stem cells, UB, WD, or any combination thereof are cultured in vitro and induced to undergo branching morphogenesis in the presence of basal conditioned media (BSN-CM) or pleiotrophin and GDNF or pleiotrphin, FGF1, and GDNF. Following propagation of the buds, the propagated buds can be recombined with mesenchyme (MM) derived from or have a phenotype associated with the branching tissue to be developed. The tissue can then undergo vascularization in vivo upon implantation within a subject. Using the various component pieces a functional branched epithelial organ can be obtained.

The disclosure demonstrates that UBs, WDs, or UBs and WDs undergo branching tubulogenesis in the presence of a conditioned medium elaborated by a cell line derived from the MM or isolated from an E11.5 mouse (BSN cells). Soluble factors present in BSN-CM promote UB and WD morphogenesis. Factors that are secreted by the MM are important for the development of the collecting system in artificial systems as well as in vivo.

The MM-derived cell conditioned medium (BSN-CM), when supplemented with GDNF, also induces the isolated UB, WD, stem cells, or UB and WD (in the absence of MM) to undergo dichotomous branching reminiscent of that seen in the developing kidney. This indicates that the MM-derived cell line, reflecting the MM itself, secretes soluble factors capable of inducing branching morphogenesis of the UB, WD, stem cells, or UB and WD. This isolated cell culture system can serve as a powerful assay system since it directly assesses the effect of soluble factors on cell morphogenesis and tubulogenesis.

The disclosure demonstrates that serial liquid column chromatographic fractionation of BSN-CM contains an active morphogenetic fraction comprising a polypeptide (capable of inducing branching morphogenesis comparable to whole BSN-CM). One such polypeptide is pleiotrophin. Pleiotrophin was originally discovered as a fibroblast proliferative factor and a neurite outgrowth-promoting factor. Outside the nervous system pleiotrophin is generally detected in those embryonic organs in which mesenchymal-epithelial interactions are thought to play an important role, such as salivary glands, lung, pancreas, and kidney.

The disclosure provides culture techniques and factors, and combination of factors capable of inducing stem cell and epithelial cells branching morphogenetic activity.

Populations of branching cells developed by the methods and compositions of the disclosure can be culture in biocompatible matrices or gels used in tissue engineering. Similarly, mesenchymal cells can be cultured in biocompatible matrices or gels. Furthermore, co-culture of mesenchymal cells (from a desired tissue or having a particular phenotype) and branching-bud cells can be co-cultured in biocompatible matrices or gels. The biocompatible matrix or gel may be designed to promote branching or directional branching (e.g., by photolithography techniques, printing techniques and the like; see, e.g., Nelson at al. (Science 314, 298 (2006)), incorporated herein by reference).

Alternatively, the stem cells or epithelial bud progenitor cells of the disclosure may be seeded onto or into a three-dimensional framework or scaffold alone (e.g., as a homogenous population) or in combination (e.g., a heterogeneous population) and cultured to allow the cells to grow and fill the matrix or immediately implanted in vivo, where the seeded cells will proliferate. Such a framework can be implanted in combination with any one or more growth factors, drugs, additional cell types, or other components that stimulate tissue (e.g., kidney tissue) formation or otherwise enhance or improve the practice of the disclosure.

The cell compositions of the disclosure can be used to produce new branching tissue (e.g., breast, salivary, pancreatic, biliary tissue and the like) in vitro, which can then be implanted, transplanted or otherwise inserted to replace or augment a subject's tissue. Upon implantation, the tissue can become vascularized. In a non-limiting embodiment, the cells are cultured to produce a three-dimensional tissue construct in vitro by combining the epithelial or endothelial buds with mesenchymal cells having a desired morphology or derived from a particular tissue to direct the growth of a desired tissue-type (e.g., breast tissue etc.), which are then implanted in vivo.

A biocompatible matrix or gel may be of arty material and/or shape that allows cells to attach to it (or can be modified to allow cells to attach to it) and allows cells to grow in more than one layer. A number of different materials may be used to form the matrix, including, but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon) , thermanox (TPX), nitrocellulose, cotton, polyglycolic acid (PGA), collagen (in the form of sponges, braids, or woven threads, and the like) , cat gut sutures, cellulose, gelatin, or other naturally occurring biodegradable materials or synthetic materials, including, for example, a variety of polyhydroxyalkanoates. Any of these materials may be woven into a mesh, for example, to form the three-dimensional framework or scaffold. The pores or spaces in the matrix can be adjusted by one of skill in the art to allow or prevent migration of cells into or through the matrix material.

The three-dimensional framework, matrix, hydrogel, and the like, can be molded into a form suitable for the tissue to be replaced or repaired. For example, various techniques are known wherein a biocompatible matrix can be molded to form tubes, channels, islands, wells, and various shapes.

The stem cells, their progeny, and generated tissue of the disclosure cart be used in a variety of applications. These include, but are not limited to, transplantation or implantation of the cells either in unattached form or as attached, for example, to a three-dimensional framework or gel, as described herein.

The cells or tissue developed according to the disclosure can be administered prior to, concurrently with, or Following injection of the angiogenic factor. The presence of an angiogenic factor associated with the implanted artificial tissue will assist in vascularization of the tissue upon implantation. In addition, the cells of the disclosure may be administered immediately adjacent to, at the same site, or remotely from the site of administration of the angiogenic factor. By angiogenic factor is meant a growth factor, protein or agent that promotes or induces angiogenesis in a subject.

In another aspect of the disclosure, artificial matrices comprising biocompatible material may be used as a support for cell growth. Such matrices may be designed such that concentrations of pleiotrophin may change at desired branch points within the matrix material. In this manner, cells may grow and proliferate through the matrix and branch at locations where pieiotrophin concentrations are at a level to induce branching morphogenesis.

Using the methods and compositions of the disclosure it is possible to grow branching tubular tissues in vitro. The methods of and compositions provide for breast tissue development, pancreas tissue development, salivary gland tissue development and the like through co-culturing of non-specific branch buds with mesenchymal tissue-specific cells.

As described herein, the disclosure demonstrates that isolated stem cells or any combination thereof undergoes branching morphogenesis in vitro when exposed to several growth factors including pleiotrophin (PTN) alone or in combination with other factors including glial cell-derived neurotrophic factor (GDNF), fibroblast growth factor-1 or -7 (FGF1, FGF7) and proteins secreted by a mesenchymally derived cell line or any combination thereof. In addition, the disclosure provides methods for regulating processes that govern stem cell, UB, and WD branching morphogenesis, such as the matrix-binding requirements vis-a-vis integrin expression, the dependence of branching morphogenesis on heparin sulfate proteoglycans, and the roles of positive and negative modulators of branching. Other growth factors present in media conditioned by ureteric bud, Wolffian duct bud, or ureteric and Wolffian duct bud cells that can induce differentiation of isolated mesenchyme cultured in vitro include, for example, leukemia-inhibitory factor (LIE) and FGF2.

Subcultures of each of the components of the kidney—the ureteric bud, Wolffian duct bud, or ureteric and Wolffian duct bud and the mesenchyme allow for “staged” development of an artificial organ tissue. Using the methods and compositions of the disclosure the isolated UB, WD, or UB and WD and mesenchyme can be recombined in vitro and grown in an autonomous fashion. The resultant organ tissue is morphologically and architecturally indistinguishable from a “normal” organ and can be used for transplantation, as a source for the study of organ function, and as a resource for determining drug-effects upon organ function. Furthermore, the disclosure provides methods for partitioning/propagating the organ or the cultured isolated ureteric bud, Wolffian duct bud, or ureteric and Wolffian duct bud into smaller fragments and support drain vitro development of these sub fractions through several “generations” (e.g., see FIG. 9A-C). The methods of the disclosure further allow for these subfractions to be recombined with fresh mesenchyme to develop additional organ tissue through the induction of the mesenchyme. Furthermore, these nascent nephrons formed contiguous connections with limbs of the branched UB, WD, or UB and WD. Consequently, the disclosure provides in vitro engineered organ tissue comprising a population of primordia suitable for transplantation and derived from a single progenitor.

The methods provided by the disclosure utilize an in vitro, approach to tissue engineering that provides an ability to create colonies of a desired tissue (e.g., pancreas tissue and in some cases comprising genetically engineered cells) suitable for transplantation. For example, in the case of pancreas tissue generation, a stem cell population, an embryonic ureteric bud, Wolffian duct bud, or ureteric and Wolffian duct bud is obtained or separated from the surrounding pancreas-specific mesenchyme and each component (e.g., the MM and UB, WD, or any combination thereof) is cultured in isolation. The stem cell, UB, WD, or combination thereof and/or the MM can modified in vitro (as described herein) in a tailored fashion to express a specific polynucleotide (e.g., a heterologous polynucleotide) or reduce expression of a specific polynucleotide to obtain a desired function (e.g., to reduce expression of immunogenic proteins). The components are then recombined to allow the morphogenesis and development of organ tissue in vitro (e.g., to generate art in vitro engineered liver). The in vitro engineered liver can then be used in transplantation, to screen for desired biological function, and/or to screen. for agents, which modulate liver function.

For example, stem cell, embryonic UB, WD, or GB and ND are dissected and separated from the surrounding tissue or metanephric mesenchyme (MM). The dissected cells are then used to grow an arborized structure, which can be subdivided into smaller fractions and used to induce additional generations of UBs, WDs, or UBs and WDs that crow and branch in vitro. The continued growth and branching is maintained in the culture. These branched “buds” can then be combined with different tissue specific mesenchyme to develop a particular tissue (e.g., lung, kidney, salivary etc.). For example, the subfraction of UBs, WDs, or UBs and WDs can then be used through multiple generations to renew kidney tissue development by recombining the kidney “buds” with kidney derived mesenchyme (e.g., metanephric mesenchyme). For example, UB, WD, or UB and WD generations can be dissected and recombined with freshly isolated metanephric mesenchyme. The cells retained the ability to induce dramatic tubular epithelial differentiation of the mesenchyme. Furthermore, there appeared to be connections between induced tubules of the mesenchyme and terminal portions of the UB, WD, or UB and WD thereby providing a conduit between the tubule and urinary collecting system. The generated kidney opens up the possibility of uniquely tailoring specific components of either the nephron (derived from the mesenchyme) or tie collecting system (derived from the UB, WD, or UB and WD) in vitro in a potentially functional and transplantable organ.

The source of cells used to ultimately engineer organ tissue need not be derived from the particular organ per se (see, e.g., Kim and Dressler (J Am Soc Nephrol 16: 3527-3534, (2005)); incorporated herein by reference). Pluripotent embryonic stem (ES) cells and pluripotent embryonic germ (EG) cells can serve as progenitor cells for a variety of differentiated cell types and recent work with human ES and EG. cells has opened the door to some potential beneficial therapeutics. When cultivated in vitro, human ES and EG cells form 3-dimensional aggregates called embryoid bodies (EB) that can then differentiate into derivatives of all three primary germ cell layers. Furthermore, these EB can be induced to differentiate into specific but different cellular components such as UBs, WDs, or UBs and WD based on conditioning by certain growth factors, such as FGF and TGF-beta. Cells derived from ES and EC cells can organize and can display a diverse set of functional properties. Finally, multipotent adult bone marrow-derived mesenchymal stem cells (MSC) may serve as an adult source of stem cells readily available for engineering of tissues derived from mesenchyme. Within the context of the kidney, cells derived from the bone marrow were found to repopulate or regenerate a variety of renal territories, including the glomerular podocyte and mesangium, interstitium, and renal epithelial tubule. Recent work suggests that there may exist one or more self-renewing “renal stem cells” found within the MM that can differentiate into the myofibroblasts of the renal stroma and/or endothelium. In addition, renal tubular progenitor cells cart be obtained using the techniques as described by Maeshima et al. (J Am Soc Nephrol 17: 188-198, (2006)) incorporated herein by reference,

As discussed herein, the disclosure provides methods and compositions whereby isolated budding epethelia (e.g., UBs, WDs, or UBs and WDs) can be co-cultured and stimulated by extrinsic factors to induce branching. These branching buds can then be combined with a tissue specific mesenchymal cell population to develop a desired tissue (e.g., combined with metanephric mesenchyme to produce kidney tissue). For example, whole isolated intact UB, WD, or UB and ND (cleanly separated from surrounding MM) can be induced to undergo branching morphogenesis in vitro in a manner similar to UB, WD, or UB and WD culture. Suspension of the isolated UBs, WDs, or UBs and WDs within, or on, a natural or artificial biocompatible substrate (e.g., Matrigel/collagen gel) and when exposed to a mixture of mesenchyme-cultured media augmented with GDNF, results in the isolated unbranched UB, WD, or UB and WD rapidly forming a polarized, extensively branched structure with an internal lumen. As described further herein, pleiotrophin, which induced branching of stem cells, UB, WD, or any combination thereof, also induces branching morphogenesis of the whole ureteric bud, Wolffian duct bud, or ureteric and Wolffian duct bud. This modulation is typically branch-promoting, elongation promoting, or branch-inhibiting. For example, FGF1 induced the formation of elongated stern cells, UB, ND, or any combination thereof branching stalks whereas FGF7 induced amorphous buds displaying nonselective proliferation with little distinction between stalks and ampullae. TGF-beta, which inhibits branching in several cell-culture model systems, also appears to inhibit the branching of the isolated stem cells, UB, WD, or any combination thereof. Endostatin, which is a cleavage product or: collagen XVIII normally found in the UB, WD, or UB and WD basement membrane, also selectively inhibits branching of the UB, WD, or UB and WD. Growth factors, such as LIF, have been isolated from UB, WD, or UB and WD conditioned media and induce mesenchymal-to-epithelial transformation of cultured mesenchyme. Other factors, such as FGF2, appear to promote survival but not differentiation of mesenchyme.

The branching isolated ureteric bud, Wolffian duct bud, or ureteric and Wolffian duct bud retains the ability to induce freshly isolated mesenchyme when recombined in vitro without exogenous growth factors. By removing the surrounding biocompatible matrix from the cultured UB, WD, or UB and WD and placing mesenchyme in close proximity, the UB, WD, or UB and WD continues to grow and extend branches into the surrounding mesenchyme. Furthermore, the mesenchyme condenses in areas where the UB, WD, or UB and WD has extended branches, and then epithelializes in a manner similar to normal branched tissue development (e.g., kidney development). This has wide-ranging implications for in vitro engineering, including the ability to independently culture ureteric bud, Wolffian duct bud, or ureteric and Wolffian duct bud and metanephric mesenchyme, modify their phenotypes in vitro, and then recombine them. For example, it may be possible to develop art engineered pancreas with properties such as enhancing insulin secretion by in vitro modification of glucose transporters or calcium ion channels, and improved digestive enzyme production and secretion. The disclosure demonstrates that these recombined “in vitro engineered organs,” comprised of cultured isolated UB, WD, or UB and WD and freshly isolated mesenchyme, form cohesive intact tubular conduits. That is, the nascent tubular nephron, derived from MM, has a tubular lumen in direct connection with the tubular lumen of the collecting system, derived from the UB, WD, or UB and WD.

The culture system and methods of the disclosure provide toe ability to propagate the isolated UB, WD, or UB and WD in vitro through several generations. For example, isolated stem cells, UB, WD, or any combination thereof are cultured in vitro and induced to undergo branching morphogenesis in the presence of BSN-CM or pleiotrophin and GDNF or pleiotrphin, FGF1, and GDNF. After 8 days, the cultured bud is subdivided into approximate 3rds and re-suspended within a suitable biocompatible matrix (matrigel/collagen gel). This 2nd generation bud is further subdivided after another 8 days of culture and the 3rd generation bud is cultured for 8 days (thus yielding at least 9 subdivided buds from one progenitor bud) (e.g., see FIG. 9A-B). These subsequent clonal generations of cultured UB, WD, or UB and WD retain the ability of the progenitor bud to induce mesenchyme upon in vitro recombination. The buds also retained the capacity to form cohesive conduits with the mesenchymally-derived tubules that they induced. Thus, the disclosure provides the ability to develop and propagate a clonal, expanded, and long-lived colony of ureteric bud, Wolffian duct bud, or ureteric and Wolffian duct buds, derived from a single progenitor bud that retains the properties of the progenitor. Using similar techniques with the MM, it is possible to develop colonies of mesenchyme derived from a single progenitor mesenchyme that can then be recombined with a propagated UB, WD, or UB and WD.

Whole embryonic organs can be propagated in a similar manner in vitro. After culturing these organs for 3 days, it is possible, using the methods of the disclosure, to subdivide into approximate 3rds the whole cultured organ and then propagate the subsequent generations in vitro. At least 3 generations, yielding 9 organs, can be generated from a single progenitor organ using the methods and compositions of the disclosure. Thus, the methods and compositions of the disclosure provide the ability for expansion of syngeneic rudiments in vitro prior to transplantation into suitable hosts.

In many tissue-engineering technologies, an extrinsic biocompatible scaffold is required to provide orientation and support for the developing tissue. In one sense, the native polymeric basement membrane (BM) is a bioactive scaffold directing the normal development of the organ. BM constituents such as endostatin can directly influence branching of the UB, WD, or UB and WD, and other components, such as HSPGs, can indirectly regulate growth by binding and releasing growth factors. The bioartificial scaffolds used in tissue engineering can be synthetic or biologic and contain or can be coated with ECM constituents, such as collagen or proteoglycans. Exciting new techniques in materials science are emerging that allows these scaffolds to be impregnated with drugs, proteins, or even DNA, and thus may be more biologically relevant. By combining a truly bioactive scaffold with cultured pluripotent cells, such as ES cells, or multipotent cells, such as MSCs or other progenitor cells derived from the mesenchyme, it may be possible to coordinate inductive signals required to derive/engineer an organ such as the kidney. For example, by varying the concentration of factors at points where branching is desired, it is possible to design a tissue having a predicted number of branch point.

Biocompatible support materials (biocompatible scaffolds) for culturing organ cells include any material and/or shape that (a) allows cells to attach to it (or can be modified to allow cells to attach to it); and (b) allows cells to grow in more than one layer. A number of different materials may be used as a culture support, including, but not limited to, nylon (polyamides), dacron (polyesters) , polystyrene, polypropylene, polyacrylates, polyvinyl compounds (e.g., polyvinylchloride), polycarbonate (PVC), polytetrafluorethylene (PTFE; teflon), thermanox (TPX), nitrocellulose, cotton, polyglycolic acid (PGA), cat gut sutures, cellulose, gelatin, dextran, collagen, decellulularized tissue (both allogenic and xenogeneic), and like.

In one aspect of the disclosure a ureteric bud, Wolffian duct bud, or ureteric and Wolffian duct bud is used as a bioactive scaffold, which could then serve as a biologically-relevant orchestrator (conductor) of the complex inductive signals that underlie “normal” organ development. In an organ such as the lung, where development is dependent on coordinated interactions between epithelium and mesenchyme, utilization of a biologically active epithelial or endothelial scaffold to induce proper differentiation, maturation and integration of surrounding multi potent cells may provide a unique opportunity to modify specific cellular functions in vitro but yet to retain the complex organizational direction required to develop a mature organ. This principle is applicable to engineering of a variety of organs, including kidney, liver, pancreas, salivary gland or breast. For example, when stem cells, UB, WD, or any combination thereof, are co-cultured with lung mesenchyme, the generated tissue expresses surfactant protein. Accordingly, the methods and compositions serve as a scaffold for a number of novel “chimeric” organs, such as an organ that comprises both pancreas and liver tissue. In should therefore be understood that all manner and types “chimeric” tissues and organs can be produced, based upon the use of two or more organ-specific mesenchymes. The ability to independently culture and then combine mesenchymally-derived elements with epithelial-derived or endothelial-derived elements allow for the integration of cellular and organ-based approaches to tissue engineering. This approach would allow one to modify cell-based elements in vitro to possess certain desirable properties but still take advantage of an organ-based approach to tissue engineering.

By culturing tissue derived from stem cells, UB, WD, or UB and WD and MM in vitro, the disclosure provides the unique opportunity to modulate each of their component functions in a site-specific manner. For example, transfection of the mesenchyme with constructs expressing organic ion transporters would lead to increased capability to handle drugs and toxins, insertion of genes coding for growth factors, such as insulin-like growth factor (IGF), would lead to markedly enhanced in vitro engineered kidney development and improved functionality, insertion of immunomodulatory elements, such as repressors of co-stimulatory molecules, could be used to improved immune tolerance; stimulation of branching in the UB, WD, or UB and WD can lead to an increased number of resultant nephrons and improved renal functionality. Thus, there are numerous of ways to design an in vitro engineered kidney with tailored function. Furthermore, by subcloning UBs, WDs, or UBs and WDs, the disclosure provides the potential to develop a large number of organs derived from a single progenitor, thus removing concerns surrounding limited supply of transplantable tissue. Third, it is possible to create a chimeric organ using the UB, WD, or UB and WD as a scaffold and recombining the UB, WD, or UB and WD with heterologous mesenchymal cells. These mesenchymal cells could be derived from embryonic stern cells that, when exposed to signals from the UB, WD, or UB and WD induce differentiation of the mesenchymal cells into epithelial or endothelial tissues. In normal adults, stem cells originating in the bone marrow repopulate portions of the kidney and differentiate into renal cells, and it is likely that embryonic stem cells also posses this ability.

While the disclosure provides for the use of branching agents to induce branching in the cells disclosed herein (e.g., endothelial, epithelial, stem cells, progenitor cells or primary cells), it should be understood that the methods disclosed herein can be used equally as well with generating non-branched tissue and organs by withholding these branching agents. Furthermore, such non-branching tissue and organs can also be generated by using cells (e.g., endothelial, epithelial, stem cells, progenitor cells or primary cells) that normally do not give rise to branched tissue in vivo, such as cells from non-branched ducts.

The approach provided by the methods and compositions of the disclosure, whereby in vitro engineered tissue developed and/or are designed to possess specific functions, such as improved immune tolerance or enhanced tubular secretion of substrate, offer original approaches to transplantation and organ therapy. Furthermore, creating clonal populations of in vitro engineered tissue creates the potential for development, of organ propagation from a single tissue. This approach is applicable to epithelial tissues such as lung, salivary glands, pancreas and the like.

The methods provided by the disclosure allow for the development of colonies of subcloned in vitro engineered tissue that have been specifically tailored to express certain functions, and are immune-naive, particularly where the tissue is derived from stem cells. Immune naive means that the cells lack “self” identification as the cells were fetally or stem cell derived and therefore should be immune tolerant.

Methods of transfecting and transforming cells are known and commonly performed in the art. Methods for transfecting/transforming kidney cells are also known, including a method for the in vivo gene transfer into the rat kidney as is described in Tomita at al. (Biochem. and Biophys. Res. Comm. 136:129-134, (1992)) which disclosure is incorporated herein. Many of the methods utilize HVJ (Sendai virus) and liposome methodology. As provided in these methods, plasmid DNA and a nuclear protein are co-encapsulated in liposomes and later co-introduced into cells. SV40 Large T antigen was the reporter gene utilized. The gene transfer can be performed by inserting a cell or culture of organ tissue with a liposome suspension. Transfection/transformation efficiency of the organ cells can be assayed by detecting SV40 large T antigen immunohistochemically. The use of a particular cationic liposome DNA mixture to deliver genes with high efficiency into a vast number of endothelial cells in a rat is described in Zhu at al. (Science 261:209-11, (1993)), which disclosure is incorporated herein, while art adenoviral-mediated gene transfer into a kidney has also been described in Moullier et al. (Kidney International 45:1220-1225, (1994)), which disclosure is also incorporated herein.

As used herein, the term “transfect” or “transform” refers to the transfer of genetic material (e.g., DNA or RNA) of interest via a vector into cells of a mammalian organ or tissue (e.g., kidney/renal tissue). The vector will typically be designed to infect a mammalian cell (e.g., a kidney cell). The genetic material of interest encodes a product (e.g., a protein polypeptide, peptide or functional RNA) whose production by a mammalian cell is desired. For example, the genetic material of interest can encode a hormone, receptor, enzyme or (poly)peptide of therapeutic value. Examples of genetic material of interest include, but are not limited to, DNA encoding cytokines, growth factors and other molecules which function extra cellularly such as chimeric toxins, e.g., a growth factor such as interleukin-2 (IL-2) fused to a toxin, e.g., the pseudomonas exotoxin, dominant negative receptors (soluble or transmembrane forms), truncated cell adhesion or cell surface molecules with or without fusions to immunoglobulin domains to increase their half-life (e.g., CTLA4-Ig). For example, transformation/transfection efficiency can be evaluated by measuring the expression of a gene product encoded by the transfected/transformed genetic material prior to and post transfection/transformation of the cells of an organ or a tissue. Ideally, the gene product is not normally expressed by the cells. Alternatively, infection of the cells of an organ or a tissue may result in an increased production of a gene product already expressed by those cells or result in production of a gene product (e.g., an antisense RNA molecule) which decreases production of another, undesirable gene product normally expressed by the cells of that organ or tissue. Generally, the genetic material encodes a gene product, which is the desired gene product to be supplied to the cells of that organ or tissue. Alternatively, the genetic material encodes a gene product, which induces the expression of the desired gene product by the cells of that organ or tissue (e.g., introduced genetic material encodes a transcription factor which induces the transcription of the gene product to be supplied to the subject). Furthermore, the genetic material could simply contain a polynucleotide, e.g., in the form of single stranded DNA to act as an antisense nucleotide. Typically, the genetic material which is used to transfect/transform a cell of an organ or a tissue via a vector is in a form suitable for expressing a gene product encoded by that genetic material in a mammalian cell. Accordingly, the genetic material includes coding and regulatory sequences required for transcription of a gene (or portion thereof) and when the gene product is a protein or peptide, translation of the gene product encoded by the genetic material. Regulatory sequences which can be included in the genetic material include promoters, enhancers and polyadenylation signals, as well as sequences necessary for transport of an encoded protein or peptide, for example N-terminal signal sequences for transport of proteins or peptides to the surface of the cell or for secretion, or for cell surface expression or secretion preferentially to the luminal or basal side. Enhancers might be ubiquitous or tissue or cell specific or inducible by factors in the local environment, e.g., inflammatory cytokines.

As used herein, the term an “effective amount” refers to a level of expression of a heterologous polynucleotide transfected or transformed into a cell, which brings about at least partially a desired therapeutic or prophylactic effect in an organ or tissue transformed by the method of the disclosure. For example, expression of genetic material of interest can then result in ate modification of the cellular activities, e.g., a change in phenotype, in an organ or a tissue that has been transformed by the method of the disclosure. In one embodiment, an effective amount of the expression of a heterologous genetic material of interest results in modulation of cellular activity in a significant number of cells of art organ transfected or transformed with the heterologous polynucleotide. A “significant number” refers to the ability of the vector to infect at least about 0.1% to at least about 15% of the cells (e.g., UBs, WDs, or UBs and WDs). Typically, at least about 5% to at least about 15% of the cells are transfected/transformed. Most commonly, at least about 10% of the cell are transfected/transformed.

A vector refers to a polynucleotide molecule capable of transporting another nucleic acid to which it has been linked into cells. Examples of vectors that exist in the art include: plasmids, yeast artificial chromosomes (YACs) and viral vectors. However, the invention is intended to include such other forms of vectors which serve equivalent functions and which become known in the art subsequently hereto.

The efficacy of a particular expression vector system and method of introducing genetic material into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulator y elements and vector to be used. The reporter gene encodes a gene product, which is easily detectable and, thus, can be used to evaluate the efficacy of the system. Standard reporter genes used in the art include genes encoding β-galactosidase, chloramphenicol acetyl transferase, luciferase and human growth hormone.

The method of the disclosure can be used to infect cells to obtain designer branched epithelial or endothelial tissues (e.g., genetically engineered kidney cells). As used herein, the term “organ cells” is intended to including UB, WD, or UB and WD and MM cell types as well as the other 15 different cell types, e.g., glomerular cells, mesangial cells, interstitial cells, tubular cell, endothelial cells, are intended to be encompassed by the term “organ cells”.

The method of the disclosure can also be used to transform a tissue generated ex vivo. For example, in a transplant setting, an organ, such as a kidney, is engineered by the methods of the disclosure, the “in vitro engineered organ” is then perfused (e.g., the collecting ducts) with a vector carrying genetic material of interest.

One potential application of the disclosure is an allograft or xenograft tissue transplantation. In this aspect, tissue generated by the methods and compositions of the disclosure are transfected/transformed with an agent (e.g., delivered to MM cells and/or UBs, WDs, or UBs and UBs) that results in organ tolerance or might help in the post-operative period for decreasing the incidence of early transplant rejection or function (e.g., due to acute tubular necrosis). Either the organ can be made less immunogenic so as to reduce the number of host T cells generated and/or the endothelial cells (e.g., endothelial cells derived from MM) can be altered so as to prevent: the adhesion/transmigration of primed immune T-cells or killer effector T-cells (e.g., by use of IL-2-toxin fusion proteins). Moreover, genes transfected/transformed into in vitro engineered tissue, such as nitric oxide synthetase (NOS), prior to transplantation could also serve to protect the organ post transplantation. These strategies make clinical sense since it is well known that early rejection episodes and malfunction lead to a worse long-term graft survival. Therefore, prevention of acute rejection and preservation of function immediately post transplant are of particular importance. Delivery of the genetic material (i.e., the heterologous polynucleotides) for this purpose can be done using methods known in the art including utilizing an adenovirus vector, lipofection, or other techniques known in the art. In addition to the heterologous polynucleotides mentioned above, these vectors can carry additional sequences comprising anti-sense constructs to one or more cell adhesion molecules (involved, in lymphocyte homing) or dominant negative constructs to these molecules, or antisense constructs to MHC antigens in the transplant or locally immune suppressive lymphokines such as interleukin-10 (IL-10) or viral IL-10 or chimeric toxins which would preferentially kill T-cells, e.g., IL-2 toxin fusion protein. It is also possible that one could interfere with the recognition part of the immune system by, for example, the local secretion of CTLA4-IgG fusion proteins. This list of candidate polynucleotides is not exhaustive. Those skilled in the art of transplantation would be expected to know of others. The genes could be delivered with constitutive promoters or with appropriate inducible enhancers.

In another aspect of the disclosure, the cell cultures (UB, WD, or UB and WD alone, MM alone, and co-cultures thereof) may be used in vitro to screen a wide variety of compounds, such as cytotoxic compounds, growth/regulatory factors, pharmaceutical agents, and the like to identify agents that modify organ function and/or cause cytotoxicity and/or organ cell death or modify organ cell proliferative activity. Examples of such agents or compounds include growth factors, peptides, and small, organic molecules in another aspect, the cells can be genetically engineered and the culture implanted in vivo, whereby screening can be measured by detecting changes in the culture using a genetically engineered label. In this aspect, vascularization can assist in providing information on the effect an agent has on tissue.

To this end, the cultures (e.g., stem cells, UB, WD, or UB and WD primary cells, UB, WD, or UB and WD cell lines, MM cells, whole organ cultures, MM/spinal cord co-cultures, and UB, WD, OR UB AND WD/MM co-cultures) are maintained in vitro and exposed to the compound to be tested. The activity of a cytotoxic compound can be measured by its ability to damage or kill cells in culture or by its ability to modify the function of cells (e.g., UB, WD, or UB and WD proliferative capacity, branching capacity, MM epithelialization capacity, particular gene expression, cell size, cell morphology, protein expression, and the like). This may readily be assessed by vital staining techniques, ELISA assays, immunohistochemistry, PCR, microarray analysis, and the like. The effect of growth/regulatory factors on the primary cells (e.g., UBs, WDs, or UBs and WDs, MMs) may be assessed by analyzing the cellular content of the culture, e.g., by total cell counts, and differential cell counts, including the number of branch points. This may be accomplished using standard cytological and/or histological techniques including the use of immunocytochemical techniques employing antibodies that define type-specific cellular antigens. The effect of various drugs on normal cells cultured in the culture system may be assessed. For example, UB, WD, or UB and WD primary cells or cell lines may be cultured in vitro under conditions that stimulate branching morphogenesis/tubulogenesis (e.g., in the presence of BSN-CM, pleiotrophin, or pleiotrophin+other factors). A test compound is then contacted with the culture and the effect the test compound has on branching morphogenesis/tubulogenesis can be compared to a control, wherein a difference is indicative of a compound that increases or decreases branching morphogenesis.

The cytotoxicity to cells (e.g., human UBs, WDs, or UBs and WDs and co-cultures of MM and UBs, WDs, or UBs and WDs) of pharmaceuticals, environmental pollutants, anti-neoplabtic agents, carcinogens, food additives, and other substances may be tested by utilizing the culture system of the invention.

First, a stable, growing tissue culture comprising branching cells and mesenchyme UB, WD, or UB and WD and/or MM cells) is established. Then, the culture is exposed to varying concentrations of a test agent. After incubation with a test agent, the culture is examined by phase microscopy to determine the highest tolerated dose—the concentration of test agent at which the earliest morphological abnormalities appear. Cytotoxicity testing can be performed using a variety of supravital dyes to assess cell viability in the culture system, using techniques known to those skilled in the art.

Once a testing range is established, varying concentrations of the test agent can be examined for their effect on viability, growth, and/or morphology of the different cell types constituting the culture by means well known to those skilled in the art.

Similarly, the beneficial effects of drugs may be assessed using the culture system in vitro; for example, growth factors, hormones, drugs which enhance tissue formation, or activity (e.g., branching activity) can be tested. In this case, stable cultures may be exposed to a test agent. After incubation, the cultures may be examined for viability, growth, morphology, cell typing, and the like as art indication of the efficacy of the test substance. Varying concentrations of the drug may be tested to derive a dose response curve.

The culture systems and/or tissues/organoids created by methods of the disclosure may be used as model systems for the study of physiologic or pathologic conditions. For example, the by using the culture systems or tissues created by the methods disclosed herein, one can model the effects of drugs and toxins in a medium or high throughout put manner, including in 2D or 3D. Moreover, physiological studies can be performed with culture systems and tissues/organoids produced by the methods of the disclosure. In a specific embodiment of the invention, the culture system arid/or tissues/organoids produced by the methods of the disclosure can be optimized to act in a specific functional manner by modifying the genome of the cells.

The tissue culture system of the disclosure may also be used to aid in the diagnosis and treatment of malignancies and diseases. For example, a biopsy of a tissue may be taken from a subject suspected of having a malignancy Of other disease or disorder of the tissue. The biopsy cells can then be separated (e.g., UBs, WDs, or UBs and WDs from MM cells etc.) and cultured according to the methods of the disclosure. For example, UBs, WDs, or UBs and WDs from the subject can be co-cultured with normal mesenchyme (e.g., heterologous MM cells) to determine biological function of the UBs, WDs, or UBs and WDs compared to UBs, WDs, or UBs and WDs derived from a normal organ. Similarly mesenchymal cells from the subject can be cultured with normal branching epithelial cells (e.g., UBs, WDs, or UBs and WDs) to examine mesenchymal cell function and activity. In addition, such cultures obtained from biopsies can be used to screen agent that modify the activity in order to identify a therapeutic regimen to treat the subject. For example, the subject's culture could be used in vitro to screen cytotoxic and/or pharmaceutical compounds in order to identify those that are most efficacious; i.e. those that kill the malignant or diseased cells, yet spare the normal cells. These agents could then be used to therapeutically treat the subject.

Where in vitro engineered tissue is generated according to the methods and compositions of the disclosure transplantation of the tissue can be performed as follows. Surgery is performed on the recipient subject to expose the tissue to be treated or site of the implantation (e.g., one or both kidneys). The in vitro engineered tissue is implanted directly into/adjacent to the recipient subject's tissue to result in the formation of chimeric tissue (e.g., an engineered tissue and natural tissue).

When implanted into the recipient's body, an incision, large enough to receive the in vitro engineered tissue is made. The location of the incision can be anywhere in a viable portion of the recipient's tissue.

The implanted in vitro engineered tissue is allowed to grow within the recipient under conditions that allow the tissue to vascularize. Suitable conditions may include the use of pre- or post-operative procedures to prevent rejection of the implant as well as the administration of factors (e.g., pleotrophin, FGF1, GNDF, and the like) that stimulate tubulogenesis and/or morphogenesis of the in vitro engineered tissue. Immunosuppression techniques (in the absence or combined with genetically engineered techniques) such as cyclosporin A (CSA) to prevent rejection of the donor tissue are known in the art.

A number of embodiments of the invention have been described. Nevertheless, it will be understood that various modifications may be made without departing from the spirit and scope of the invention. Accordingly, other embodiments are within the scope of the following claims.

1. A method of generating an organ specific tubular tissue structure, comprising:
(a) contacting a stem cell, branching epithelial cell, or branching endothelial cell with one or more cell survival agents or biological active agents to stimulate growth and proliferation;
(b) contacting the cells with one or more branching agents that promotes formation of tubular tissue branches and/or globular morphology to generate budding tissue;
(c) combining the bud tissue with tissue specific mesenchyme in a biocompatible matrix; and
(d) culturing the combination to form an organ specific tubular tissue structure from which the tissue specific mesenchyme was obtain in vitro.

2. The method of claim 1, wherein the branching epithelial cell is a ureteric bud, bud or duct of a salivary gland, Wolffian duct bud, or ureteric and Wolffian duct bud tissue.

3. The method of claim 1, wherein the organ specific mesenchyme is from the group consisting of breast, pancreatic, lung, gall bladder, spleen, liver, and glandular mesenchyme.

4. The method of claim 3, wherein the glandular mesenchyme is selected from the group consisting of adrenal, salivary, prostate, thymus, parathyroid, pituitary, and pineal mesenchyme.

5. The method of claim 1, wherein the one or more cell survival agents are selected from the group consisting of FGF1, FGF7 and a combination thereof either alone or in combination with one or more of GDNF, PTN, HRG, or BSN-CM.

6. The method of claim 5, the one or more cell survival agents are selected from the group consisting of FGF1, FGF7, FGF1 and FGF7, PTN and GDNF, FGF1 and GDNF, FGF7 and GDNF, BSN-CM and FGF1, HRG and FGF1, PTN and FGF1, BSN and FGF7, HRG and FGF7, PTN and FGF7, BSN and FGF1 and GDNF, HRG and FGF1 and GDNF, PTN and FGF1 and GDNF, BSN and FGF7 and GDNF, HRG and FGF7 and GDNF, and PTN and FGF7 and GDNF.

7. The method of claim 1, wherein the one or more branching agents are selected from the group consisting of Fgf1, Fgf2, Fgf7, Fgf10, Hgf, Tgfα, Activin, Bmp4, Tgfβ, Gremlin1, ErbB/neuregulin/heregulin, Fgfr2, Egfr, Pax2, Eya1, Six1, Wnt4, Wnt5a, Wnt11, B-Catenin, Pea3/Etv4, Hs2st, MMP2, MT1-MMP, Fibronectin, Notch, Sonic Hedgehog, Sprouty1, Sprouty2, SFRP1, Semaphorin3a, Slit/Robo, and Eph/Ephrin.

8. The method of claim 1, wherein the biocompatible matrix comprises a material selected from the group consisting of a cotton, a collagen, a polyglycolic acid, a cat gut suture, a cellulose, a gelatin, a dextran, a polyamide, a polyester, a polystyrene, a polypropylene, a polyacrylate, a polyvinyl, a polycarbonate, a polytetrafluorethylene, a nitrocellulose compound, and a Matrigel.

9. The method of claim 8, wherein the biocompatible matrix is treated to contain proteoglycans, Type I collagen, Type IV collagen, laminin, proteoglycans, fibronectin, or combinations thereof.

10. The method of claim 1, further comprising culturing the budding tissue in vitro under conditions that induce branching morphogenesis to generate a population of tubular branches; and subdividing the population of tubular branches; and re-suspending each subpopulation in culture media and repeating.

11. A breast, pancreatic, lung, gall bladder, spleen, liver, reproductive, glandular or vascular tissue produced by the method of claim 1.

12. The method of claim 11, wherein the glandular tissue is selected from the group consisting of adrenal, salivary, prostate, thymus, parathyroid, pituitary, and pineal.

13. The tissue of claim 11, wherein the tissue is implanted into a subject so as to induce vascularization of the tissue.

14. A method to treat a disease, disorder or condition in a subject comprising implanting the tissue of claim 11 or a portion thereof in a subject.

15. The method of claim 14, wherein the disease, disorder or condition is selected from the group consisting of Sjogren syndrome, Addison's disease, Celiac disease, chronic thyroiditis, multiple sclerosis, systemic lupus erythematosus, diabetes, pancreatitis, hypertension, chronic kidney disease, polycystic kidney disease, end stage renal disease, malignant hypertension, acute liver failure, chronic liver failure, chronic hepatitis infection, liver cirrhosis, hemochromatosis, Wilson's disease, nonalcoholic steatohepatitis, hepatocellular carcinoma, hepatoblastoma, cholangiocarcinoma, biliary artesia, coronary artery disease, cardiomyopathy, heart failure, cystic fibrosis, emphysema, obstructive lung disease, short bowel syndrome, necrotizing enterocolitis, and Crohn's disease.

16. A method to reconstruct tissue that has been removed from a subject comprising:
implanting the tissue of claim 11 or a portion thereof in a subject to replace damaged tissue that has been removed from the subject.

17. The method of claim 16, wherein the tissue that is replaced is breast tissue.

18. A test kit comprising a tissue of claim 11.

19. A method comprising:
differentiating stem cells to form tissue specific mesenchymal cells;
differentiating stem cells to form epithelial bud cells; and
combining the cells in a biocompatible matrix or gel; and
culturing the combination to form a specific tissue.
